SEQUENCE LISTING (51 sequences)

SEQ ID NO: 1 - protein, 624 aa, Homo sapiens
MENSDSNDKGSGDQSAAQRRSQMDRLDREEAFYQFVNNLSEEDYRLMRDNNLLGTPGESTEEELLRRLQQIKEGPPPQNSDENRGGDSSDDVSNGDSIIDWLNSVRQTGNTTRSGQRGNQSWRAVSRTNPNSGDFRFSLEINVNRNNGSQNSENENEPSARRSSGENVENNSQRQVENPRSESTSARPSRSERNSTEALTEVPPTRGQRRARSRSPDHRRTRARAERSRSPLHPMSEIPRRSHHSISSQTFEHPLVNETEGSSRTRHHVTLRQQISGPELLSRGLFAASGTRNASQGAGSSDTAASGESTGSGQRPPTIVLDLQVRRVRPGEYRQRDSIASRTRSRSQTPNNTVTYESERGGFRRTFSRSERAGVRTYVSTIRIPIRRILNTGLSETTSVAIQTMLRQIMTGFGELSYFMYSDSDSEPTGSVSNRNMERAESRSGRGGSGGGSSSGSSSSSSSSSSSSSSSSSSSSPSSSSGGESSETSSDLFEGSNEGSSSSGSSGARREGRHRAPVTFDESGSLPFLSLAQFFLLNEDDDDQPRGLTKEQIDNLAMRSFGENDALKTCSVCITEYTEGNKLRKLPCSHEYHVHCIDRWLSENSTCPICRRAVLASGNRESVV

SEQ ID NO: 2 - RNA, 15 nt, Artificial Sequence
  Description of Artificial Sequence Synthetic Rlim_XM_ 006527919_1959_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
aggcaacaaacuuca

SEQ ID NO: 3 - RNA, 15 nt, Artificial Sequence
  Description of Artificial Sequence Synthetic Rlim_XM_ 006527919_2724_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
uauuuaucuugugga

SEQ ID NO: 4 - RNA, 15 nt, Artificial Sequence
  Description of Artificial Sequence Synthetic Rlim_XM_ 006527919_2747_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
aaagccuaugcuuga

SEQ ID NO: 5 - RNA, 15 nt, Artificial Sequence
  Description of Artificial Sequence Synthetic Rlim_XM_ 006527919_2772_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
aaaaucacauucaua

SEQ ID NO: 6 - RNA, 15 nt, Artificial Sequence
  Description of Artificial Sequence Synthetic Rlim_XM_ 006527919_2815_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
aggcucaguacuuua

SEQ ID NO: 7 - RNA, 15 nt, Artificial Sequence
  Description of Artificial Sequence Synthetic Rlim_XM_ 006527919_2831_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
caaugcacuguugua

SEQ ID NO: 8 - RNA, 15 nt, Artificial Sequence
  Description of Artificial Sequence Synthetic Rlim_XM_ 006527919_3562_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
cauguuggguuauua

SEQ ID NO: 9 - RNA, 15 nt, Artificial Sequence
  Description of Artificial Sequence Synthetic Rlim_XM_ 006527919_4070_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
aaggaaauaauguca

SEQ ID NO: 10 - RNA, 15 nt, Artificial Sequence
  Description of Artificial Sequence Synthetic Rlim_XM_ 006527919_4084_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
caaacaaagcuucaa

SEQ ID NO: 11 - RNA, 15 nt, Artificial Sequence
  Description of Artificial Sequence Synthetic Rlim_XM_ 006527919_4137_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
cgcuuauguaauaca

SEQ ID NO: 12 - RNA, 15 nt, Artificial Sequence
  Description of Artificial Sequence Synthetic Rlim_XM_ 006527919_4140_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
uuauguaauacauua

SEQ ID NO: 13 - RNA, 15 nt, Artificial Sequence
  Description of Artificial Sequence Synthetic Rlim_XM_ 006527919_6849_s siRNA
  modified_base (1)..(4): 2′-O-Methyl nucleotide
  misc_feature (1)..(3): Phosphorothioate linkage
  modified_base (5)..(7): 2′-Fluoro nucleotide
  modified_base (8)..(8): 2′-O-Methyl nucleotide
  modified_base (9)..(9): 2′-Fluoro nucleotide
  modified_base (10)..(15): 2′-O-Methyl nucleotide
  misc_feature (13)..(15): Phosphorothioate linkage
  3′ TegChol
uagguacgcaguuga

SEQ ID NO: 14 - RNA, 20 nt, Artificial Sequence
  Description of Artificial Sequence Synthetic Rlim_XM_ 006527919_1959_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
ugaaguuuguugccuucugu

SEQ ID NO: 15 - RNA, 20 nt, Artificial Sequence
  Description of Artificial Sequence Synthetic Rlim_XM_ 006527919_2724_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uccacaagauaaauaucuca

SEQ ID NO: 16 - RNA, 20 nt, Artificial Sequence
  Description of Artificial Sequence Synthetic Rlim_XM_ 006527919_2747_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
ucaagcauaggcuuuuuaau

SEQ ID NO: 17 - RNA, 20 nt, Artificial Sequence
  Description of Artificial Sequence Synthetic Rlim_XM_ 006527919_2772_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uaugaaugugauuuuuuuca

SEQ ID NO: 18 - RNA, 20 nt, Artificial Sequence
  Description of Artificial Sequence Synthetic Rlim_XM_ 006527919_2815_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uaaaguacugagccucuaca

SEQ ID NO: 19 - RNA, 20 nt, Artificial Sequence
  Description of Artificial Sequence Synthetic Rlim_XM_ 006527919_2831_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uacaacagugcauuggaaaa

SEQ ID NO: 20 - RNA, 20 nt, Artificial Sequence
  Description of Artificial Sequence Synthetic Rlim_XM_ 006527919_3562_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uaauaacccaacaugacuuc

SEQ ID NO: 21 - RNA, 20 nt, Artificial Sequence
  Description of Artificial Sequence Synthetic Rlim_XM_ 006527919_4070_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
ugacauuauuuccuugcaag

SEQ ID NO: 22 - RNA, 20 nt, Artificial Sequence
  Description of Artificial Sequence Synthetic Rlim_XM_ 006527919_4084_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uugaagcuuuguuuggacau

SEQ ID NO: 23 - RNA, 20 nt, Artificial Sequence
  Description of Artificial Sequence Synthetic Rlim_XM_ 006527919_4137_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uguauuacauaagcgccaua

SEQ ID NO: 24 - RNA, 20 nt, Artificial Sequence
  Description of Artificial Sequence Synthetic Rlim_XM_ 006527919_4140_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
uaauguauuacauaagcgcc

SEQ ID NO: 25 - RNA, 20 nt, Artificial Sequence
  Description of Artificial Sequence Synthetic Rlim_XM_ 006527919_6849_as siRNA
  5′ Phosphate
  modified_base (1)..(1): 2′-O-Methyl nucleotide
  modified_base (2)..(2): 2′-Fluoro nucleotide
  modified_base (3)..(5): 2′-O-Methyl nucleotide
  misc_feature (1)..(3): Phosphorothioate linkage
  modified_base (6)..(6): 2′-Fluoro nucleotide
  modified_base (7)..(13): 2′-O-Methyl nucleotide
  misc_feature (13)..(20): Phosphorothioate linkage
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
ucaacugcguaccuacucca

SEQ ID NO: 26 - RNA, 20 nt, Artificial Sequence
  Description of Artificial Sequence Synthetic Rlim_XM_ 006527919_1959 siRNA
acagaaggcaacaaacuucg

SEQ ID NO: 27 - DNA, 45 bp, Homo sapiens
tgcattacagaatacacagaaggcaacaaacttcgtaaactacct

SEQ ID NO: 28 - RNA, 20 nt, Artificial Sequence
  Description of Artificial Sequence Synthetic Rlim_XM_ 006527919_2724 siRNA
ugagauauuuaucuugugga

SEQ ID NO: 29 - DNA, 45 bp, Homo sapiens
aatttagagtttaattgagatatttatcttgtggaaatattaaaa

SEQ ID NO: 30 - RNA, 20 nt, Artificial Sequence
  Description of Artificial Sequence Synthetic Rlim_XM_ 006527919_2747 siRNA
auuaaaaagccuaugcuugu

SEQ ID NO: 31 - DNA, 45 bp, Homo sapiens
ttatcttgtggaaatattaaaaagcctatgcttgtgtaagtgaaa

SEQ ID NO: 32 - RNA, 20 nt, Artificial Sequence
  Description of Artificial Sequence Synthetic Rlim_XM_ 006527919_2772 siRNA
ugaaaaaaaucacauucauu

SEQ ID NO: 33 - DNA, 45 bp, Homo sapiens
ctatgcttgtgtaagtgaaaaaaatcacattcatttgtttaaaaa

SEQ ID NO: 34 - RNA, 20 nt, Artificial Sequence
  Description of Artificial Sequence Synthetic Rlim_XM_ 006527919_2815 siRNA
uguagaggcucaguacuuuu

SEQ ID NO: 35 - DNA, 45 bp, Homo sapiens
aatgtaaagctattttgtagaggctcagtacttttccaatgcact

SEQ ID NO: 36 - RNA, 20 nt, Artificial Sequence
  Description of Artificial Sequence Synthetic Rlim_XM_ 006527919_2831 siRNA
uuuuccaaugcacuguugua

SEQ ID NO: 37 - DNA, 45 bp, Homo sapiens
gtagaggctcagtacttttccaatgcactgttgtattaatgcatt

SEQ ID NO: 38 - RNA, 20 nt, Artificial Sequence
  Description of Artificial Sequence Synthetic Rlim_XM_ 006527919_3562 siRNA
gaagucauguuggguuauuu

SEQ ID NO: 39 - DNA, 45 bp, Homo sapiens
attttttattgaaaggaagtcatgttgggttatttgagattcaaa

SEQ ID NO: 40 - RNA, 20 nt, Artificial Sequence
  Description of Artificial Sequence Synthetic Rlim_XM_ 006527919_4070 siRNA
cuugcaaggaaauaaugucc

SEQ ID NO: 41 - DNA, 45 bp, Homo sapiens
aggatatgtactgctcttgcaaggaaataatgtccaaacaaagct

SEQ ID NO: 42 - RNA, 20 nt, Artificial Sequence
  Description of Artificial Sequence Synthetic Rlim_XM_ 006527919_4084 siRNA
auguccaaacaaagcuucac

SEQ ID NO: 43 - DNA, 45 bp, Homo sapiens
tcttgcaaggaaataatgtccaaacaaagcttcacttattttttt

SEQ ID NO: 44 - RNA, 20 nt, Artificial Sequence
  Description of Artificial Sequence Synthetic Rlim_XM_ 006527919_4137 siRNA
uauggcgcuuauguaauaca

SEQ ID NO: 45 - DNA, 45 bp, Homo sapiens
gcatatttcactgtttatggcgcttatgtaatacatttttatctt

SEQ ID NO: 46 - RNA, 20 nt, Artificial Sequence
  Description of Artificial Sequence Synthetic Rlim_XM_ 006527919_4140 siRNA
ggcgcuuauguaauacauuu

SEQ ID NO: 47 - DNA, 45 bp, Homo sapiens
tatttcactgtttatggcgcttatgtaatacatttttatcttttt

SEQ ID NO: 48 - RNA, 20 nt, Artificial Sequence
  Description of Artificial Sequence Synthetic Rlim_XM_ 006527919_6849 siRNA
uggaguagguacgcaguugg

SEQ ID NO: 49 - DNA, 45 bp, Homo sapiens
ctgtgaagtgtccagtggagtaggtacgcagttggcctggttata

SEQ ID NO: 50 - RNA, 21 nt, Artificial Sequence
  Description of Artificial Sequence Synthetic RLIM_4070_ p3_AS siRNA
  5′-(E)-Vinylphosphonate
  modified_base (1)..(1): 2′-O-Methyl nucleotide
  misc_feature (1)..(3): Phosphorothioate linkage
  modified_base (2)..(2): 2′-Fluoro nucleotide
  modified_base (3)..(3): 2′-O-Methyl nucleotide
  modified_base (4)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  misc_feature (14)..(21): Phosphorothioate linkage
  modified_base (15)..(15): 2′-O-Methyl nucleotide
  modified_base (16)..(16): 2′-Fluoro nucleotide
  modified_base (17)..(19): 2′-O-Methyl nucleotide
  modified_base (20)..(20): 2′-Fluoro nucleotide
  modified_base (21)..(21): 2′-O-Methyl nucleotide
ugacauuauuuccuugcaagu

SEQ ID NO: 51 - RNA, 16 nt, Artificial Sequence
  Description of Artificial Sequence Synthetic RLIM_4070_ p3_S_Dio siRNA
  modified_base (1)..(3): 2′-O-Methyl nucleotide
  misc_feature (1)..(3): Phosphorothioate linkage
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  misc_feature (14)..(16): Phosphorothioate linkage
  modified_base (15)..(16): 2′-O-Methyl nucleotide
caaggaaauaauguca

(19) United States
(12) Patent Application Publication
(10) Pub. No.: US 2022/0340903 A1
(43) Pub. Date: Oct. 27, 2022

(54) TARGETING RLIM TO MODULATE BODY WEIGHT AND OBESITY

(71) Applicant: University of Massachusetts, Minneapolis, MN (US)

(72) Inventors: Ingolf Bach, Framingham, MA (US); Feng Wang, Shrewsbury, MA (US); Anastasia Khvorova, Westborough, MA (US); Chantal Ferguson, Worcester, MA (US)

(21) Appl. No.: 17/760,504

(22) PCT Filed: Sep. 25, 2020
(86) PCT No.: PCT/US2020/052856

§ 371 Date: Mar. 15, 2022

Related U.S. Application Data
(60) Provisional application No. 62/905,694, filed on Sep. 25, 2019.

(51) Int. Cl. C12N 15/113

(52) U.S. Cl. CPC C12N 15/113; C12N 2310/312; C12N 2310/315; C12N 2310/3515

(57) ABSTRACT

Methods for the treatment of weight-related disorders, including obesity and disorders associated with obesity, as well as underweight and disorders associated with underweight, by modulating Rlim levels.

### CLAIM OF PRIORITY

This application claims the benefit of U.S. Provisional Patent Application Ser. No. 62/905,694, filed on Sep. 25, 2019. The entire contents of the foregoing are hereby incorporated by reference.

### FEDERALLY SPONSORED RESEARCH OR DEVELOPMENT

This invention was made with Government support under Grant No. GM128168 awarded by the National Institutes of Health. The Government has certain rights in the invention.

### TECHNICAL FIELD

Described herein are methods for the treatment of weight-related disorders, including obesity and disorders associated with obesity, as well as underweight and disorders associated with underweight, by modulating Rlim levels and/or activity.

### BACKGROUND

The development of obesity is driven by an imbalance in energy homeostasis, which requires the coordinated regulation of food intake and energy expenditure (Morton et al., 2014). The major control center for energy homeostasis is the hypothalamus in the brain, which consists of multiple interconnected nuclei (FIG. 1), including the arcuate (Arc), ventromedial (VMN), paraventricular (PVN), dorsomedial (DMN), lateral (LHA), and ventromedial (VMN) nuclei (Breton, 2013; Morton et al., 2014). Neurons in the preoptic area (POA) sense body temperature and project to various hypothalamic nuclei including the ventromedial nucleus (VMN). Activated by low body temperature POA neurons modulate communication within various hypothalamic regions (Contreras et al., 2017), leading to the activation of thermogenesis in particular by brown adipose tissues (BAT) via the sympathetic nervous system (SNS) (Breton, 2013; Contreras et al., 2017) (Contreras et al., 2017; Cristancho and Lazar, 2011) (see FIG. 1). In contrast to BAT, the white adipose tissue (WAT) functions as a major site of energy/lipid storage and is the primary site of synthesis of the hormone leptin, which serves as an endocrine feedback signal to regulate appetite, mediated by specific neurons in the Arc nucleus that express the leptin receptor (LepR) (Morton et al., 2014). Indeed, much of the functions of leptins on appetite are mediated by inhibitory GABAergic neurons that project to specific hypothalamic brain regions (Vong et al., 2011). Thus, the communication between nuclei within the hypothalamus integrate signals to regulate food intake and adjust the level of BAT thermogenesis, adipose stores as well as energy utilization (FIG. 1) (Breton, 2013; Contreras et al., 2017; Morton et al., 2014).

### SUMMARY

The present disclosure uncovers multiple aspects of Rlim function in balancing energy homeostasis in vivo, including the regulation of appetite/food intake as well as regulation of energy expenditure/thermogenesis. Presented findings demonstrate that mice lacking Rlim are protected from obesity. The present data provide evidence that this is the consequence of an imbalance between food intake and thermogenesis/lipid metabolism. As shown herein, the RlimKO mouse model is a paradigm for the study of how energy homeostasis is controlled, in particular how signals regulating appetite are integrated with those regulating thermogenesis by adipose tissues. Thus, interfering with the Rlim pathway may allow shifting the steady-state of the body's energy setpoint towards lower appetite but increased energy expenditure and identify novel strategies and targets for therapeutic intervention to manipulate energy metabolism. In particular, the majority of attempts of treating obesity have focused solely on reducing appetite (Dietrich and Horvath, 2012). However, drugs targeting exclusively food intake have proven inefficient because any reduction achieved has been compensated by decreased energy expenditure (Dietrich and Horvath, 2012). This renders the direct or indirect interference with the Rlim pathway in brain as a promising strategy to treat obesity.

Thus, provided herein are methods for treating, or reducing risk of, obesity or a disorder associated with obesity, or improving glycemic control, in a mammalian subject. The methods include administering a therapeutically effective amount of an inhibitory nucleic acid targeting Rlim to a subject in need thereof. Also provided herein are inhibitory nucleic acids targeting Rlim, for use in a method of treating, or reducing risk of, obesity or a disorder associated with obesity in a mammalian subject.

In some embodiments, the disorder associated with obesity is diabetes, metabolic syndrome, fatty liver disease, non-hepatic steatosis.

In some embodiments, the inhibitory nucleic acid is an antisense, siRNA, or shRNA. In some embodiments, the inhibitory nucleic acid is an siRNA comprising at least 10 consecutive nucleotides of SEQ ID NO: 2-25, 26, 28, 30, 32, 34, 36, 38, 40, 42, 46, 48, 50, or 51.

In some embodiments, the inhibitory nucleic acid comprises one or more modified bonds or bases.

In some embodiments, the one or more modified bonds or bases comprise morpholinos, phosphorothioate backbones, peptide nucleic acid (PNA), or locked nucleic acid (LNA) molecules. In some embodiments, the inhibitory nucleic acid is conjugated to a N-Acetylgalactosamine (GalNAc) and/or hydrophobic moiety. In some embodiments, the hydrophobic moiety is or comprises dichloroacetic acid (DCA) or Phosphatidylcholine (PC)-DCA, Docosahexaenoic acid (DHA), or Phosphatidylcholine-DHA (g2DHA), or cholesterol. In some embodiments, the inhibitory nucleic acid is divalent (e.g., Dio), trivalent, or tetravalent.

In some embodiments, the subject has a BMI of at least 25.

In some embodiments, the subject is human.

Also provided herein are methods for treating, or reducing risk of, underweight or a disorder associated with underweight in a mammalian subject. The methods include administering a therapeutically effective amount of an Rlim polypeptide to a subject in need thereof. Also provided herein are Rlim polypeptides or nucleic acids encoding Rlim, for use in a method of treating, or reducing risk of, underweight or a disorder associated with underweight in a mammalian subject.

In some embodiments, the subject has a BMI of less than 18.5.

In some embodiments, the subject is human.

In some embodiments, the methods include administering (i) a polypeptide comprising a sequence that is at least 95% identical to SEQ ID NO:1, or an active fragment thereof, or (ii) a nucleic acid encoding a polypeptide comprising a sequence that is at least 95% identical to SEQ ID NO:1, or an active fragment thereof.

In some embodiments, the methods include administering an inhibitory nucleic acid or a nucleic acid encoding a polypeptide comprising a sequence that is at least 95% identical to SEQ ID NO:1, in a viral vector.

In some embodiments, the viral vector is an adeno-associated viral (AAV) vector.

In some embodiments, the inhibitory nucleic acid, nucleic acid, vector, or polypeptide is administered parenterally.

In some embodiments, the inhibitory nucleic acid, nucleic acid, vector, or polypeptide is administered intravenously, intramuscularly, or subcutaneously.

In some embodiments, the polypeptide comprises one or more modifications. In some embodiments, the modification comprises one or more of replacement of one or more L amino acids with D amino acids; acetylation (e.g., comprises an N-acetylalanine at position 2), amidation; conjugation to a linear or branched-chain monomethoxy poly-ethylene glycol (PEG); modification of the N- or C-terminus; glycosylation; polysialic acid (PSA) addition to a glycan; or fusion to a non-Rlim protein.

Additionally, provided herein are inhibitory nucleic acids comprising at least 10 consecutive nucleotides of SEQ ID NO: 2-25, 26, 28, 30, 32, 34, 36, 38, 40, 42, 46, 48, 50, or 51.

In some embodiments, the inhibitory nucleic acid comprises one or more modified bonds or bases. In some embodiments, the one or more modified bonds or bases comprise morpholinos, phosphorothioate backbones, peptide nucleic acid (PNA), or locked nucleic acid (LNA) molecules. In some embodiments, the inhibitory nucleic acid is conjugated to a N-Acetylgalactosamine (GalNAc) and/or a hydrophobic moiety. In some embodiments, the hydrophobic moiety is or comprises dichloroacetic acid (DCA) or Phosphatidylcholine (PC)-DCA, Docosahexaenoic acid (DHA), or Phosphatidylcholine-DHA (g2DHA), or cholesterol. In some embodiments, the inhibitory nucleic acid is divalent (e.g., Dio), trivalent, or tetravalent.

Also provided are the inhibitory nucleic acid targeting Rlim described herein for use in a method of treating, or reducing risk of, obesity or a disorder associated with obesity in a mammalian subject, e.g., wherein the disorder associated with obesity is diabetes, metabolic syndrome, fatty liver disease, non-hepatic steatosis.

An Rlim polypeptide or nucleic acid encoding Rlim, for use in a method of treating, or reducing risk of, underweight or a disorder associated with underweight in a mammalian subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 Nervous system control of thermoregulation via the hypothalamus-adipose axis. Coronal view hypothalamic nuclei through the hypothalamus. Shown are arcuate, ventromedial, dorsomedial and paraventricular nuclei (Arc, VMN, DMN and PVN, respectively), the lateral hypothalamic area (LHA) and the nucleus of the solitary tract (NTS) in the brainstem. Communication pathways induced by cold temperature between hypothalamic nuclei are indicated (blue arrows, simplified). Neuronal signals, especially from the VMN and PVN mediate thermo-regulation e.g. by modulating the activity of sympathetic autonomic nervous system (brown arrows) via the nucleus of the solitary tract (NTS, brainstem). The hypothalamus receives feedback from brown and white adipose tissues (BAT and WAT, respectively) via leptins (red arrows). Adapted from (Breton, 2013).

FIGS. 2A-E Lack of Rlim protects from diet-induced obesity. Males carrying a germline Rlim KO (KO/Y) and control littermates (fl/Y), housed at room temperature (n>5 each genotype). Animals were fed 12 weeks with normal chow food (ND) and then switched over to High Fat diet (HFD). A) Total animal weights are shown. B) Weight of organs isolated from 32 weeks old males. C) Representative adipose tissues. D) H&E staining. Note diminished load in lipid droplets of KO/Y animals. E) KO animals display less than half the fat mass when compared to control littermates. ***=P<0.001.

FIGS. 3A-C Lack of Rlim protects from liver steatosis. Males carrying a germline Rlim KO (KO/Y) and control littermates (fl/Y), housed at room temperature (n>5 each genotype). Animals were fed 12 weeks with normal chow food (ND) and then switched over to High Fat diet (HFD). A) Representative livers. B) H&E staining. C) Staining of lipids on frozen sections via Oil Red O dye. Note diminished lipid load in KO/Y livers.

FIGS. 4A-E Male and female mice lacking Rlim are lean. Weight growth charts of animals housed at room temperature and fed with normal chow food (ND). The cKO of the maternally transmitted fl alleles in males and females was induced by a paternal Sox2-Cre transgene. A) Comparison of male fl/Y and cKO/Y^{Sox2-cre} littermates. B) Comparison of females. (KO=SC-cKOm/KOp; controls=fl/fl and flm/KOp). C) Weight of organs and D) body lengths of 10 weeks old males E) Difference in fat mass is responsible for weight difference between fl/and SC-cKO/Y animals (HFD; 1H-MRS). ***=P<0.001; **=P<0.01.

FIGS. 5A-C Lack of Rlim protects from obesity induced by aging and thermal environment. cKO/Y^{Sox2-cre} and WT/Y mice were raised at 21° C. At 8 months of age, littermates were separated and kept either at 21° C. or 29° C. for another 28 weeks. A) Weight charts, B) Organ weights, and C) Adipose tissues.

FIGS. 6A-G Imbalance of energy homeostasis in mice lacking Rlim. A) Increased heat production in scapular BAT of males systemically lacking Rlim (infrared camera). Left: example of mice on HFD; right: summary of mice on ND and HFD). B, C) Increased UCP1 mRNA levels (B) and protein (C) in BAT of mice lacking Rlim. D) Increased energy expenditure in RlimKO mice (metabolic cages). E) Mice lacking Rlim eat significantly less when compared to littermate controls. F, G) While lipid absorption appears normal in cKO/Y^{Sox2-cre} males (F), lipid levels in serum is decreased (G) (ND and HFD). TQ triglycerides; Chol, cholesterol; PL phospholipids. ***=P<0.001; **=P<0.01; *=P<0.05

FIGS. 7A-B The Rlim mouse model is relevant to obesity in humans. A) Weight chart of cKO/Y^{Sox2-cre} (red) and fl/Y littermates (black), born and raised at room temperature (21° C.; left panel) or at 29° C. (right panel) and fed HFD at 12 weeks of age. B) Weight chart of system-wide Rlim cKO induced at in adults via tamoxifen-injections using Ubc-Cre/ERT2 (red) and fl/Y control littermates (black). Results indicate that effects of the Rlim KO model are based on the disturbance of a persistent activity as opposed to developmental defects. Animals were kept at 21° C. and fed HFD at 12 weeks of age.

FIGS. 8A-F Lack of Rlim activity in nervous tissues is responsible for the lean phenotype. Weight charts of mice with the Rlim cKO induced via various Cre drivers targeting different tissues and cell types. cKO/Y and fl/Y littermates (21° C.; ND) are shown in grey and black, respectively. All Cre drivers have been tested independently. A) No weight changes in mice with the Rlim cKO induced in all adipose tissues via Adipoq-Cre. B) Rlim cKO induced in all neuronal tissues via Nestin-Cre recapitulated the phenotype observed upon systemic Rlim deletion (left panel: 21° C.; right panel: 29° C.). C-F) No effects seen on body weight in mice with Rlim targeted in excitatory (CamK2-Cre; C), dopaminergic (DAT-Cre; D), cholinergic (Chat-Cre; E) and glutamatergic (Vglut-Cre; F) neurons.

FIGS. 9A-F Lack of Rlim activity in inhibitory GABAergic neurons contributes to the lean phenotype. A) Rlim cKO induced in GABAergic neurons via Vgat-Cre recapitulated much of the lean phenotype. B) RLIM protein was detected in GABAergic neurons in the hypothalamus. 3^{rd} ventricle is indicated (V3) IHC using antibodies against RLIM on brain sections of mice expressing GFP specifically in GABAergic neurons. Note high RLIM expression in nuclei of neurons in the Arc that express cytoplasmic GFP. C-F) No effects seen on body weight in mice with Rlim targeted in POMC neurons (POMC-Cre; C) or Agrp neurons (Agrp-Cre; D), and neurons of the PVH (Sim1-Cre; E) and VMN (Vglut-Sfl; F).

FIGS. 10A-E Lack of Rlim activity in Ngn3-positive cell types contributes to the lean phenotype. A) Rlim cKO induced via Ngn3-Cre recapitulates much of the lean phenotype. B-D) No effects on body weight in mice with Rlim targeted in the pancreas (Pdx1-Cre; B) and pancreatic beta cells (Ins1-Cre; C) or to gut epithelial cells (Vil1-Cre; D) and enteroendocrine cells in the gut (CCK-Cre; E).

FIGS. 11A-B Identification of siRNAs sequences suitable for silencing Rlim in mice and humans. A) Screen of 12 fully chemically modified siRNAs targeting mouse and human Rlim in N2A cells identifies 7 functional sequences. Top Dose: 1.5 uM; 72 hour time point. B) 8-point dose response curves of hit sequences. All sequences result in potent and efficacious silencing with low IC50 values.

FIGS. 12A-D Rlim acts in the Leptin pathway. A) Generation of male mice harbouring an Rlim and Leptin (ob) double-KO. Left panel: Body weight; right panel: food intake. Note that during lactation the effect of ob/ob on food intake is fully dependent on Rlim. In adults no effect of the Rlim KO in the ob/ob background was observed both on body weight and food intake, indicating that Rlim acts downstream in the leptin pathway. These results establish a genetic link between both genes and reveal a major shift in the role of Rlim during the regulation of food intake after weaning. B) Body weight and food intake of animals with a Rlim cKO^{Nestin-Cre} an ob/ob background. Note similar patterns pattern compared to (A). C) Increased POMC expression was seen in Arcuate nuclei of animals lacking Rlim (32w; HFD) as shown by IHC (DAB staining) D) No weight changes seen in mice with the Rlim cKO induced via LepR-Cre, indicating functions of Rlim in cell types different from in Leptin receptor-positive neurons.

### DETAILED DESCRIPTION

The X-linked gene Rlim (also known as Rnf12) encodes a ubiquitin ligase (E3) (Ostendorff et al., 2002) that shuttles between the nucleus and cytoplasm (Jiao et al., 2013). While Rlim mRNA is detected in many tissues, RLIM protein expression is more restricted (Ostendorff et al., 2006) likely due to regulated autoubiquitination ability and short half-life (Becker et al., 2003). RLIM promotes both activation or repression of specific genes via proteasome-dependent adjustment of nuclear levels of transcription factors and transcriptional cofactors (Gontan et al., 2012; Gungor et al., 2007; Kramer et al., 2003; Ostendorff et al., 2002; Bach et al., 1999), including cofactors of LIM homeodomain proteins (CLIM; LDB)(Ostendorff et al., 2002). Moreover, transcriptional regulation also involves the direct recruitment of RLIM by LIM homeodomain and ERα transcription factors on promoters/enhancers, where it regulates the dynamics of transcriptional multiprotein complexes (Gungor et al., 2007; Johnsen et al., 2009; Ostendorff et al., 2002). In mice, Rlim functions as a crucial sex-specific epigenetic regulator of female nurturing tissues including the placenta and mammary gland.

In a mouse model allowing the Cre-recombinase-mediated conditional KO (cKO) of the Rlim gene (Ostendorff et al., 2000; Shin et al., 2010), it was found that the transmission of a maternal germline Rlim KO allele (KOₘ or Δₘ) to female embryos induces early embryonic lethality, due to defective implantation. This was caused by the inhibition of placental trophoblast functions due to defective imprinted X-chromosome inactivation (XCI)(Shin et al., 2010; Wang et al., 2016), an epigenetic process that adjusts X dosage compensation between genders (Disteche, 2012; Lee and Bartolomei, 2013; Payer, 2016). Moreover, RLIM serves as a survival factor specifically for milk-producing alveolar cells in mammary glands of adult pregnant and lactating females (Jiao et al., 2012; Jiao et al., 2013). Not surprisingly, RLIM protein is highly expressed in the nuclei of the cell types that exert defects upon the Rlim KO in vivo, e.g. cells of the preimplantation embryo/trophoblasts (Shin et al., 2014; Wang et al., 2016) and mammary alveolar cells (Jiao et al., 2012). In contrast to females, males with a germline Rlim KO allele (KO/Y) are born, grow into adulthood and are fertile (Shin et al., 2010; Shin et al., 2014; Wang et al., 2016; Wang et al., 2020). However, these males exhibit defects during spermiogenesis, with sperm containing excess cytoplasm, leading to decreased motility and in vitro fertilization rates (Wang et al., 2020). In mice, Rlim mRNA is widely detected, whereas RLIM protein expression is more specific in cell types and organs including the nervous system (Ostendorff et al., 2006).

As shown herein, functions of Rlim for the establishment of energy homeostasis in mice have been investigated. Because female embryos with a maternally inherited RlimKO die around implantation due to iXCI failure (Shin et al., 2010), male animals lacking Rlim in various tissues and cell types were generated.

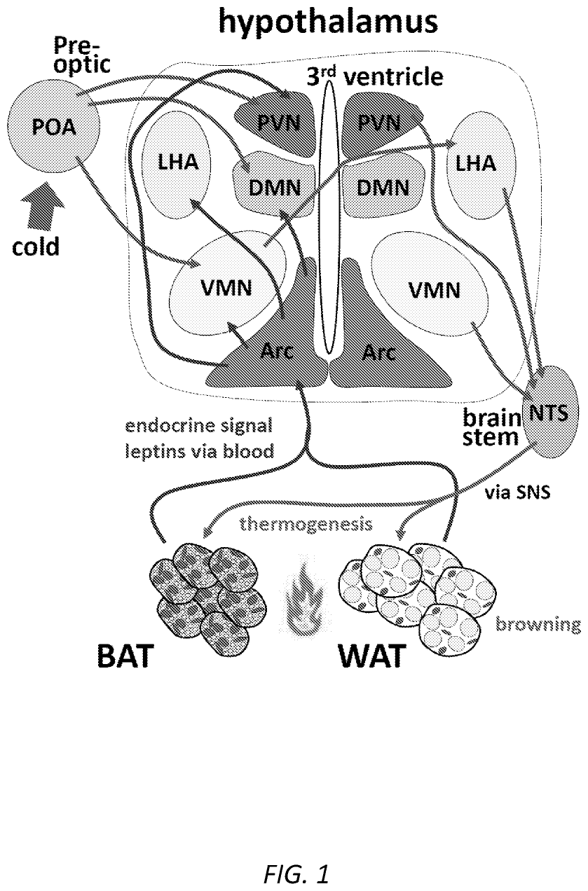

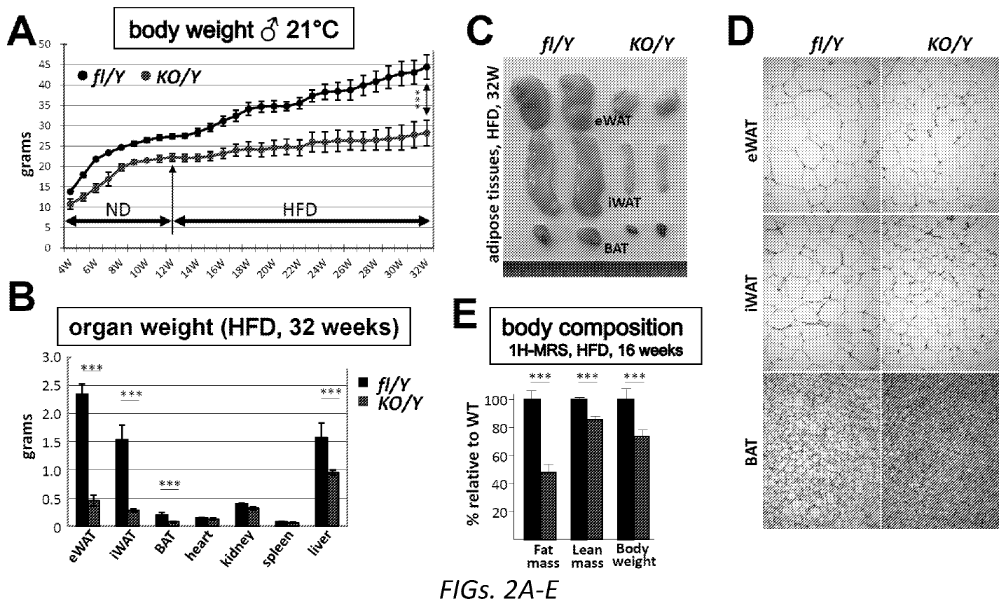

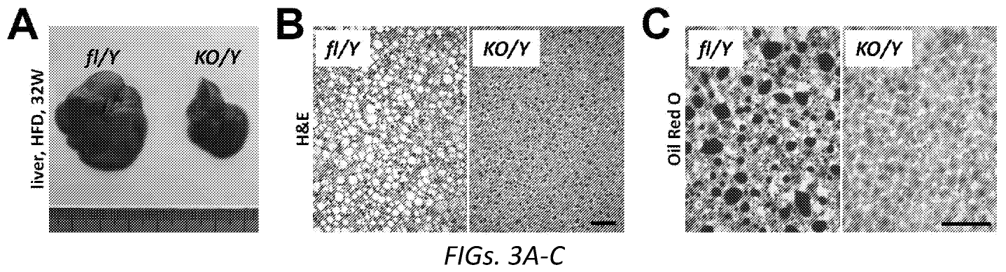

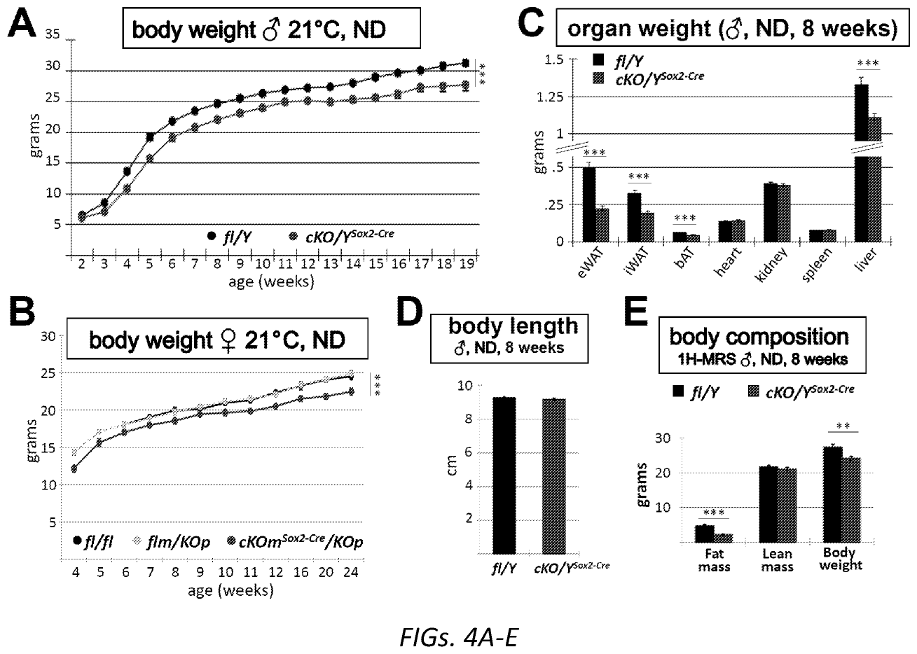

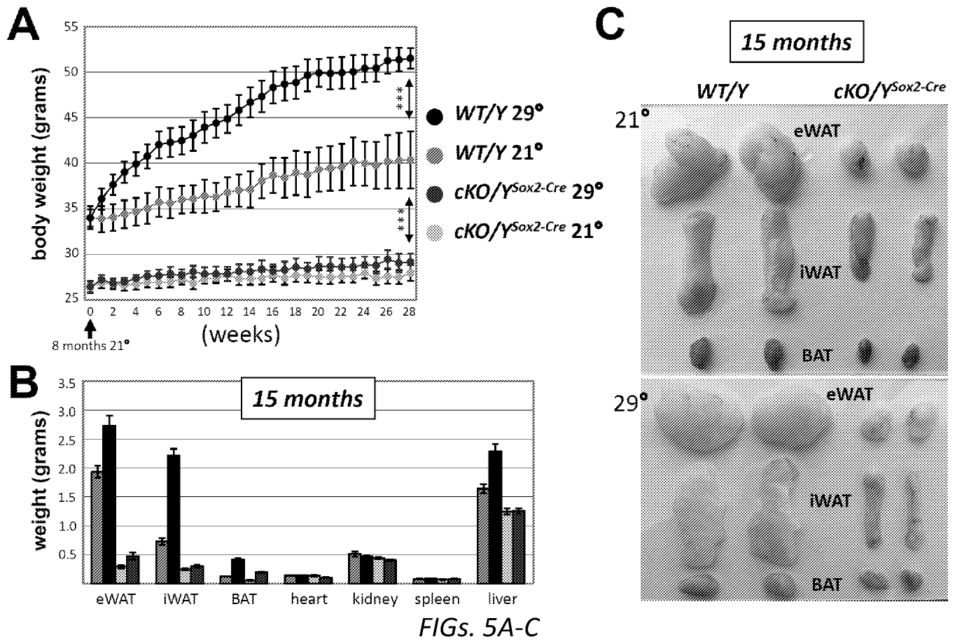

Analyses of males systemically lacking Rlim revealed that these mice are protected from obesity and development of liver steatosis. Measuring weights of animals after birth, our results indicate that males carrying a germline KO of Rlim results display significantly decreased weight than control littermates (FIG. 2A). Indeed, inducing obesity by switching diet from normal to high fat (ND; HFD, respectively) at 12 weeks of age, results reveal only gradual weight gains in males with a germline Rlim KO compared to control littermates that become obese by 32 weeks of age (FIG. 2A). Analysis of organs of animals at 32 weeks shows that adipose tissues (eWAT, iWAT and BAT) of KO animals as well as liver display significantly less weight, whereas other organs appear not affected (FIG. 2B; not shown). Indeed, eWAT, iWAT, BAT and liver are smaller with a lower load in lipid droplets (FIG. 2C, D). Moreover, comparing body composition via 1H-MRS showed that the Rlim KO mostly predominantly affect the fat mass in these animals (FIG. 2E). Focusing analyses on the liver of HFD-fed animals at 32 weeks of age revealed lower lipid load also in this organ as revealed by weight, size, histology and Oil Red O staining (FIGS. 1B, 2A-C). This effect was not sex-specific as both males and females systemically lacking Rlim via a Sox2-Cre (Hayashi et al., 2002; Hayashi et al., 2003) targeted cKO weighed significantly less than their control littermates (FIGS. 4A, B), with lower lipid loads in adipose tissues and liver but similar body lengths (FIGS. 4C-E). Thus, because HFD-fed control but not Rlim KO animals develop obesity within the recorded time period, these results indicate that mice lacking Rlim are protected from diet-induced obesity as well as liver steatosis. We next examined obesity induced by age and thermal environment by investigating 8 months-old males that were housed at 21° C. (ND). Animals were split, and half of animals were either housed at 29° C. (ND) or maintained at 21° C. (ND). While control animals quickly gained weight and became obese, those systemically lacking Rlim (Sox2-Cre cKO/Y) did not, even when housed at 29° C. (FIG. 5A-C). Combined, these results reveal that mice lacking Rlim are protected from obesity induced by diet, age and/or thermal environment.

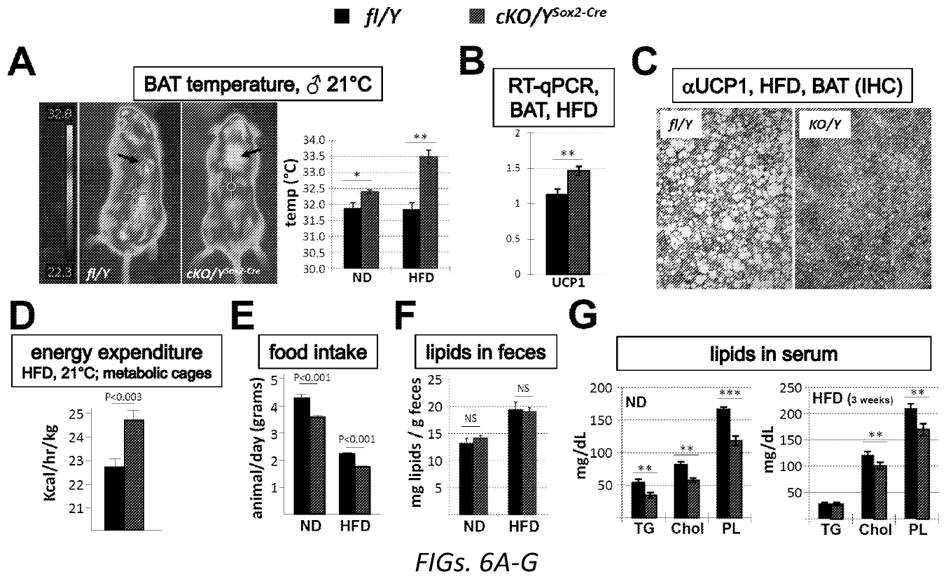

We next tested the underlying causes for the lean phenotype of animals lacking Rlim. Testing temperature of the BAT in live animals using a thermal camera, we found significantly higher temperature of the scapular BAT in mice lacking Rlim (FIG. 6A), consistent with elevated UCP1 mRNA and protein levels in this tissue (FIGS. 6B and C, respectively) and overall energy expenditure was increased (FIG. 6D). Importantly, we also found significantly lower food intake of both ND and HFD in animals lacking Rlim when compared to control littermates, (FIG. 6E), and while absorption of lipids in the food was normal (FIG. 6F), serum levels of lipids including triglycerides, cholesterol and phospholipids were decreased (FIG. 6G). Thus, animals lacking Rlim displayed increased lipid metabolism in BAT and eat less than control littermates, indicating defects in the integration of energy expenditure and food intake.

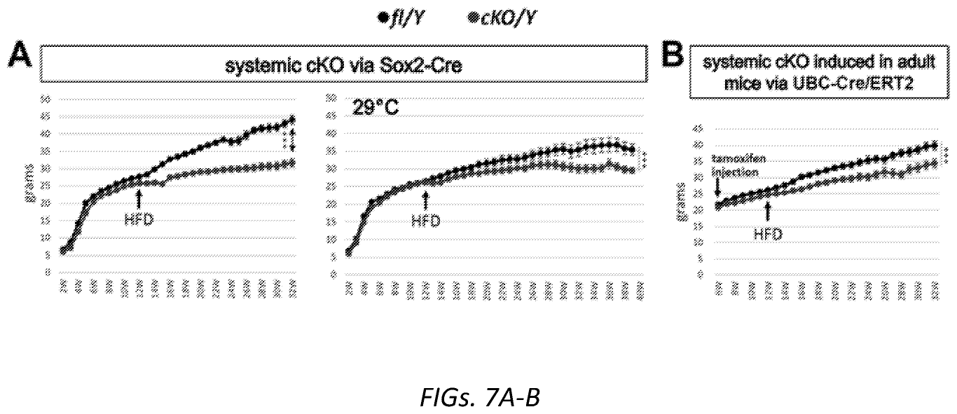

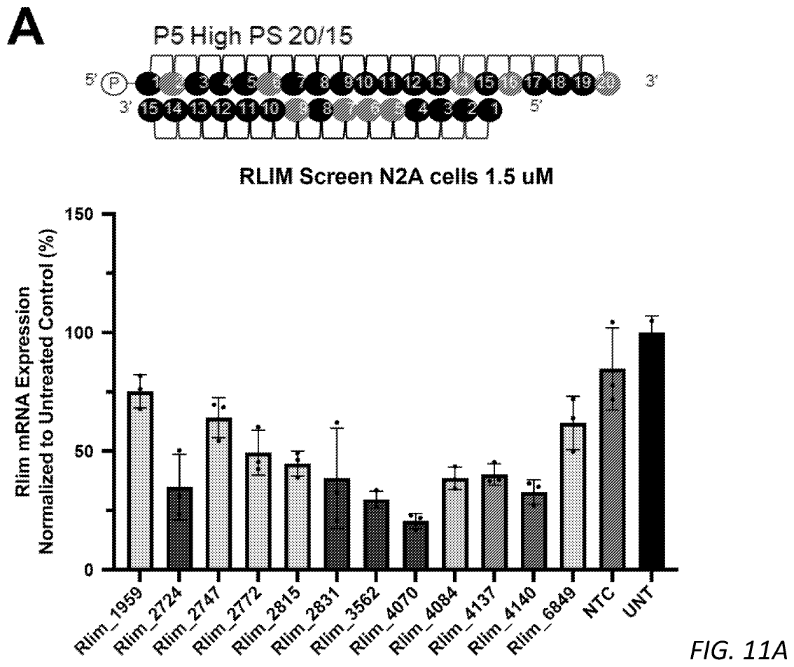

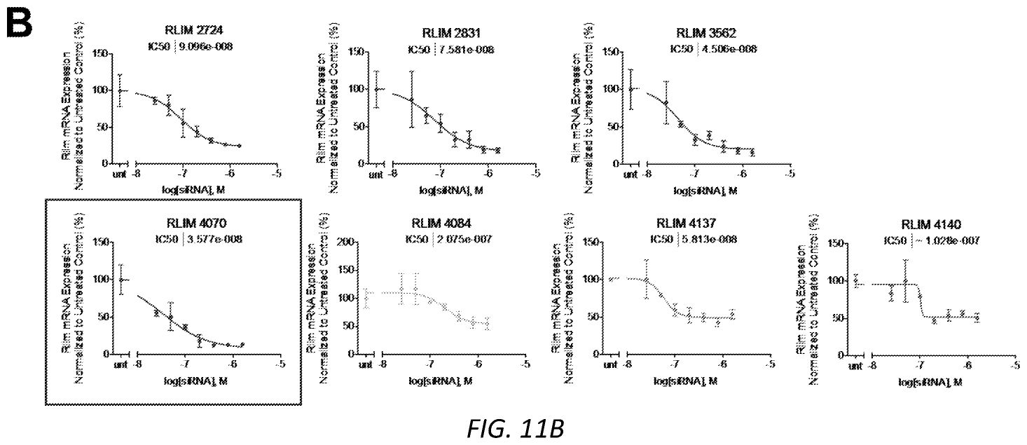

Because housing mice at thermoneutrality is thought to best mimic the thermal environment of humans (Fischer et al., 2018), we also confirmed the weight effect of the RlimKO in animals born and housed at 29° C. (FIG. 7A). To determine if the Rlim KO phenotype is based on a developmental defect, we used UBC-cre^{ERT2} mice (Ruzankina et al., 2007) to induce a systemic Rlim cKO in adult mice at 6 weeks of age. Our finding that the above observed phenotype gradually establishes after tamoxifen injections in Rlim UBC-cre^{ERT2} cKO but not in control-injected littermates (and UBC-cre^{ERT2}-only controls), indicates that the Rlim KO phenotype is caused by an activity of Rlim that is persistent in adult animals (FIG. 7B). We screened 12 chemically modified siRNAs of Rlim sequences conserved between mouse and human in N2A cells and identified 7 that led to an efficient Rlim knockdown (KD) (FIG. 11A,B). Because chemically modified siRNAs have been used for silencing genes in vivo (Alterman et al., 2019; Biscans et al., 2018), our combined results render the Rlim model suitable for research on intervention/treatment of obesity in humans.

Combined, our data identify the Rlim gene pathway as a novel model for studying the regulation of balancing energy homeostasis with major implications in the fields of energy metabolism, food intake and obesity. Our results provide evidence that the Rlim pathway could be used as prime target for the development of novel drugs against obesity.

### Methods of Treatment

The methods described herein include methods for the treatment of obesity and disorders associated with obesity, e.g., diabetes and metabolic syndrome. In some embodiments, the disorder is diet-induced obesity, e.g., high-calorie or high-fat diet induced obesity. Generally, the methods include administering a therapeutically effective amount of an inhibitory nucleic acid targeting Rlim as described herein, to a subject who is in need of, or who has been determined to be in need of, such treatment.

As used in this context, to “treat” means to ameliorate at least one symptom of obesity or a disorder associated with obesity. Often, obesity results in hyperglycemia; thus, a treatment can result in a reduction in blood glucose levels and a return or approach to normoglycemia, and/or a reduction in BMI. Administration of a therapeutically effective amount of a compound described herein for the treatment of obesity will result in decreased body weight or fat.

Alternatively, the present methods can be used to increase weight, e.g., in subjects who are underweight (e.g., to treat low weight or lack of appetite). In this context, administration of a therapeutically effective amount of a compound described herein for the treatment of underweight will result in increased body weight or fat.

RLIM Polypeptides

Exemplary sequences for human Rlim are known in the art; see, e.g., GenBank Ref. no. NM_016120.3 (nucleic acid, variant 1), which encodes NP_057204.2 (protein), and NM_183353.2 (nucleic acid, variant 2), which encodes NP_899196.1 (protein). Variant 1 lacks an alternate exon in the 5′ UTR, as compared to variant 2, and may represent the more abundant form. Variant 2 represents the longer transcript. Both variants encode the same protein. The following is an exemplary sequence of RLIM (NP_057204.2):

| (SEQ ID NO: 1) | |
|---|---|
| 1 | MENSDSNDKG SGDQSAAQRR SQMDRLDREE AFYQFVNNLS EEDYRLMRDN NLLGTPGEST |
| 61 | EEELLRRLQQ IKEGPPPQNS DENRGGDSSD DVSNGDSIID WLNSVRQTGN TTRSGQRGNQ |
| 121 | SWRAVSRTNP NSGDFRFSLE INVNRNNGSQ NSENENEPSA RRSSGENVEN NSQRQVENPR |
| 181 | SESTSARPSR SERNSTEALT EVPPTRGQRR ARSRSPDHRR TRARAERSRS PLHPMSEIPR |
| 241 | RSHHSISSQT FEHPLVNETE GSSRTRHHVT LRQQISGPEL LSRGLFAASG TRNASQGAGS |
| 301 | SDTAASGEST GSGQRPPTIV LDLQVRRVRP GEYRQRDSIA SRTRSRSQTP NNTVTYESER |
| 361 | GGFRRTFSRS ERAGVRTYVS TIRIPIRRIL NTGLSETTSV AIQTMLRQIM TGFGELSYFM |
| 421 | YSDSDSEPTG SVSNRNMERA ESRSGRGGSG GGSSSGSSSS SSSSSSSSSS SSSSSSPSSS |
| 481 | SGGESSETSS DLFEGSNEGS SSSGSSGARR EGRHRAPVTF DESGSLPFLS LAQFFLLNED |
| 541 | DDDQPRGLTK EQIDNLAMRS FGENDALKTC SVCITEYTEG NKLRKLPCSH EYHVHCIDRW |
| 601 | LSENSTCPIC RRAVLASGNR ESW |

The Rlim compositions used in the methods described herein can include a polypeptide that is at least 80%, e.g., at least 85%, 90%, or 95% identical to the amino acid sequence of SEQ ID NO:1, e.g., have differences at up to 5%, 10%, 15%, or 20% of the residues of SEQ ID NO:1 replaced, e.g., with conservative mutations, or deleted. Alternatively, the compositions can include nucleic acids that encode a polypeptide that is at least 80%, e.g., at least 85%, 90%, or 95% identical to the amino acid sequence of SEQ ID NO:1, e.g., have differences at up to 5%, 10%, 15%, or 20% of the residues of SEQ ID NO:1 replaced, e.g., with conservative mutations, or deleted. The variants useful in the present methods retain a desired activity of the parent, e.g. the proteasomal targeting of specific proteins such as Rex1 and CLIM/Ldb as well as functions for X chromosome inactivation and the survival of milk-producing alveolar cells in female mammary glands.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. See, e.g., Altschul et al. (2005) FEBS J. 272:5101-5109. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present invention, the comparison of sequences and determination of percent identity between two sequences can be accomplished using a Blosum62 scoring matrix with a gap penalty of 11,1.

In some embodiments, the protein includes one or more modifications, e.g., is acetylated (e.g., comprises an N-acetylalanine at position 2), amidated, conjugation to either linear or branched-chain monomethoxy poly-ethylene glycol (PEG, i.e., PEGylation), modification of the N- or C-terminus, glycosylation, polysialic acid (PSA) addition to a glycan, or fusion proteins, e.g., Fc fusion proteins, fusion to human serum albumin, fusion to carboxy-terminal peptide, and other polypeptide fusion approaches to make drugs with more desirable pharmacokinetic profiles; see, e.g., Werle and Bernkop=Schnurch, Amino Acids. 2006 June; 30(4):351-67; Strohl, BioDrugs. 2015; 29(4): 215-239.

Inhibitory Nucleic Acids

In some embodiments, the methods include administering one or more inhibitory nucleic acids targeting RLIM. Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target Rlim nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11,12, 13,14, 15,16, 17,18, 19,20,21,22, 23,24,25,26,27,28,29,30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence). In some embodiments, the inhibitory nucleic acids comprise at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of SEQ ID NO: 2-25, 26, 28, 30, 32, 34, 36, 38, 40, 42, 46, 48, 50, or 51.

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.10% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Small RNAs (siRNA; shRNA) that lead to an efficient knock-down of Rlim in cells have been described (see Ostendorff et al., 2002; Johnsen et al., 2009; Jiao et al., 2013).

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009).

In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)-O—CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, amino sugars, e.g., N-Acetylgalactosamine (GalNAc), an amino sugar derivative of galactose (see Springer and Dowdy, Nucleic Acid Therapeutics. 109-118 (2018) http://doi.org/10.1089/nat.2018.0736); hydrophobic moieties such as a lipid, e.g., cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), aphospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), apalmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, the inhibitory nucleic acid is a conjugate as described in PCT/US2017/046593, e.g., a hydrophobically modified siRNA (hsiRNA) conjugate comprising a variety of hydrophobic modifications, including conjugations to dichloroacetic acid (DCA) or Phosphatidylcholine (PC)-DCA, Docosahexaenoic acid (DHA), or Phosphatidylcholine-DHA (g2DHA). See, e.g., Osborn et al., Nucleic Acids Res. 2019 Feb. 20; 47(3): 1070-1081. In addition, the inhibitory nucleic acid can be modified with multivalent fatty acids, for example divalent myristic acid (see, e.g., Biscans, J Control Release. 2019 May 28; 302: 116-125. In addition, the valency of the inhibitory nucleic acid itself might be increased to include divalent, tetravalent and trivalent configurations (see Alternman et al., Nat Biotechnol. 2019 August; 37(8): 884-894), comprising a plurality of inhibitory nucleic acids, e.g., two, three, or four (or more) fully chemically modified, phosphorothioate-containing siRNAs, connected with a linker, e.g., a tetra-ethylene glycol (TEG) linker. These configurations showed enhanced distribution to a range of tissues, including liver and fat. Finally, the structural and chemical configuration of the inhibitory nucleic acid, e.g., the siRNA, itself can be modified. In addition to the use of the cleavable linkers, the structure of the inhibitory nucleic acid (siRNA) and its PS content may be varied. When targeting liver use of low PS content and a two base pair overhang might be optimal. When targeting fat, use of blunt configurations might be of benefit (see Biscans et al., Nucleic Acids Res. 2020 Aug. 20; 14(3): 7665-7680). For silencing of genes in the brain, the inhibitory nucleic acid can be a conjugate as described in Alterman et al., 2019, e.g. a divalent (Dio)-hsiRNA. In some embodiments, the conjugate is a trivalent N-acetylgalactosamine (GalNAc)-hsiRNA conjugate (see Osborn et al., Nucleic Acids Res. 2019 Feb. 20; 47(3): 1070-1081).

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2-modified nucleotide, e.g., a 2′-deoxy, 2′-deoxy-2′-fluoro, 2′-O-methyl, 2′-O-methoxyethyl (2′-O-MOE), 2-O-aminopropyl (2-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I Theory and Nucleic Acid Preparation, Tijssen, ed. Elsevier, N.Y. (1993).

### Pharmaceutical Compositions

The methods described herein can include the administration of pharmaceutical compositions and formulations comprising inhibitory nucleic acid sequences designed to target an RLIM RNA (e.g., to treat obesity or related disorders), or Rlim polypeptides (e.g., to treat low weight or lack of appetite).

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21 st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21 st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST % 45, ALT % 35) for all doses with the exception of the 75 mg/kg dose of miR-122 ASO, which showed a very mild increase in ALT and AST levels. They concluded that 50 mg/kg was an effective, non-toxic dose. Another study by KrOtzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

### Diabetic and Pre-Diabetic Subjects

In some embodiments, the subjects treated by the methods described herein have diabetes, i.e., are diabetic. A person who is diabetic has one or more of a Fasting Plasma Glucose Test result of 126 mg/dL or more; a 2-Hour Plasma Glucose Result in a Oral Glucose Tolerance Test of 200 mg/dL or more; and blood glucose level of 200 mg/dL or above. In some embodiments, the subjects treated by the methods described herein are being treated for diabetes, e.g., have been prescribed or are taking insulin, meglitinides, biguanides, thiazolidinediones, or alpha-glucosidase inhibitors.

In some embodiments the subjects are pre-diabetic, e.g., they have impaired glucose tolerance or impaired fasting glucose, e.g., as determined by standard clinical methods such as the intravenous glucose tolerance test (IVGTT) or oral glucose tolerance test (OGTT), e.g., a value of 7.8-11.0 mmol/L two hours after a 75 g glucose drink for impaired glucose tolerance, or a fasting glucose level (e.g., before breakfast) of 6.1-6.9 mmol/L.

The pathogenesis of type 2 diabetes is believed to generally involve two core defects: insulin resistance and beta-cell failure (Martin et al., Lancet 340:925-929 (1992); Weyer et al., J. Clin. Invest. 104:787-794 (1999); DeFronzo et al., Diabetes Care. 15:318-368 (1992)). Important advances towards the understanding of the development of peripheral insulin resistance have been made in both animal models and humans (Bruning et al., Cell 88:561-572 (1997); Lauro et al., Nat. Genet. 20:294-298 (1998); Nandi et al., Physiol. Rev. 84:623-647 (2004); Sreekumar et al., Diabetes 51:1913-1920 (2002); McCarthy and Froguel, Am. J. Physiol. Endocrinol. Metab. 283:E217-E225 (2002); Mauvais-Jarvis and Kahn, Diabetes. Metab. 26:433-448 (2000); Petersen et al., N. Engl. J. Med. 350:664-671 (2004)). Thus, those subjects who have or are at risk for insulin resistance or impaired glucose tolerance are readily identifiable, and the treatment goals are well defined.

In some embodiments, the methods described herein include selecting subjects who have diabetes or pre-diabetes. In some embodiments, the following table is used to identify and/or select subjects who are diabetic or have pre-diabetes, i.e., impaired glucose tolerance and/or impaired fasting glucose.

Fasting Blood Glucose

| | |
|---|---|
| From 70 to 99 mg/dL (3.9 to 5.5 mmol/L) | Normal fasting glucose |
| From 100 to 125 mg/dL (5.6 to 6.9 mmol/L) | Impaired fasting glucose |
| | (pre-diabetes) |
| 126 mg/dL (7.0 mmol/L) and above on more | Diabetes |
| than one testing occasion | |

Oral Glucose Tolerance Test (OGTT) [except pregnancy]
(2 hours after a 75-gram glucose drink)

| | |
|---|---|
| Less than 140 mg/dL (7.8 mmol/L) | Normal glucose tolerance |
| From 140 to 200 mg/dL (7.8 to 11.1 | Impaired glucose tolerance |
| mmol/L) | (pre-diabetes) |
| Over 200 mg/dL (11.1 mmol/L) on more | Diabetes |
| than one testing occasion | |

Body Mass Index (BMI)

Obesity increases a subject's risk of developing T2D. BMI is determined by weight relative to height, and equals a person's weight in kilograms divided by height in meters squared (BMI=kg/m²). Accepted interpretations are given in Table 3.

TABLE 3

| Category | BMI |
|---|---|
| Underweight | ≤18.5 |
| Normal weight | 18.5-24.9 |
| Overweight | 25-29.9 |
| Obese | ≥30 |

Thus, the methods described herein can include determining a subject's height, determining a subject's weight, and calculating BMI from the values determined thereby. Alternatively, the methods described herein can include reviewing a subject's medical history to determine their BMI.

In some embodiments, the methods described herein include selecting subjects who have a BMI of 30 or above (i.e., obese subjects).

Metabolic Syndrome

In some embodiments, the methods include determining whether a subject has the metabolic syndrome, and selecting the subject if they do have the metabolic syndrome, then administering an inhibitory nucleic acid as described herein. Determining whether a subject has the metabolic syndrome can include reviewing their medical history, or ordering or performing such tests as are necessary to establish a diagnosis.

The metabolic syndrome, initially termed Syndrome X (Reaven, Diabetes. 37(12):1595-1607 (1988)), refers to a clustering of obesity, dyslipidemia, hypertension, and insulin resistance. All components of the metabolic syndrome are traditional risk factors for vascular disease. As used herein, the metabolic syndrome is defined by the presence of at least 3 of the following: abdominal obesity (excessive fat tissue in and around the abdomen, as measured by waist circumference: e.g., greater than 40 inches for men, and greater than 35 inches for women), fasting blood triglycerides (e.g., greater than or equal to 150 mg/dL), low blood HDL (e.g., less than 40 mg/dL for men, and less than 50 mg/dL for women), high blood pressure (e.g., greater than or equal to 130/85 mmHg) and/or elevated fasting glucose (e.g., greater than or equal to 110 mg/dL). In some embodiments, levels of these criteria may be higher or lower, depending on the subject; for example, in subjects of Asian ancestry; see, e.g., Meigs, Curr. Op. Endocrin. Diabetes, 13(2):103-110 (2006). A determination of the presence of metabolic syndrome can be made, e.g., by reviewing the subject's medical history, or by reviewing test results.

Based on data from the Third National Health and Nutrition Examination Survey (NHANES III) approximately 24% of the adults in the United States qualify as having the metabolic syndrome (Ford et al., JAMA. 287(3):356-359 (2002)). Insulin resistance is now felt to be central in the pathogenesis of these related disorders.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1. Rlim as a Target for Protection from Obesity

To further analyze functions of Rlim, we generated animals with a systemic cKO of Rlim induced at blastocyst stages via a paternally transmitted Sox2-Cre transgene. Because Cre expression is induced after iXCI, this strategy allows for the generation of both males and females that lack systemic Rlim (Shin et al., 2014). Indeed, both males or females with an Sox2-Cre-induced Rlim cKO display decreased weight in adipose tissues when compared to their control littermates (FIGS. 4A-C; 7A). The observed consequences in animals with Sox2-Cre-mediated deletion of Rlim were largely recapitulating the phenotype observed in germline KO animals (compare with FIG. 2) with slightly less efficiency, likely due to incomplete penetrance of the Cre driver, and the presence of the Sox2-Cre transgene alone had no effect on animal weight (not shown). Indeed, Rlim cKO/Y^{Sox2-cre} and control animals displayed similar lengths (FIG. 4D), and 1H-MRS analyses revealed that the difference in weights of 8 weeks old animals was solely due to differences in fat mass (FIG. 4E). We also found evidence of browning of adipose tissues in cKO/Y animals and higher levels of UCP1, in particular in BAT (FIGS. 6B, C). Consistent with this was the finding of elevated body temperature, in particular in animals on HFD (FIG. 6A). Measurements using metabolic cages confirmed an elevated energy expenditure in cKO animals (FIG. 6D) and decreased food intake and blood lipid levels (FIGS. 6E, G). These changes occurred without significant changes in nutrient absorption or physical activity (FIG. 6F, and data not shown).

Mice kept at 21° C. are slightly cold-stressed. As thermoneutral conditions are thought to better reflect human conditions, we also tested the effects of the Rlim mutation on adipose tissues under thermoneutral conditions (29°). Because our results indicated that lack of Rlim protects from HFD-induced obesity (FIGS. 2; 3), we first tested protection from obesity caused by thermoneutral conditions. Rlim cKO mice and control littermates were raised under ND at 21° C. At 8 months of age, littermates were separated and kept either at 21° C. or 29° C. for another 32 weeks. While control littermates developed obesity, cKO/Y littermates were protected, even at the advanced age of 15 months (FIG. 5). Thus, mice lacking Rlim were protected from obesity induced by either HFD, age or change of temperature. Next, we tested mice born and raised at thermoneutral conditions (29° C.). While we did not detect significant differences in weight gain between cKO/Y and control littermates under ND, shifting to HFD at 12 weeks of age induced significantly stronger weight gains in control animals when compared to cKO/Y littermates (FIG. 7A), indicating that the RlimKO mouse model is relevant to human obesity and associated pathologies.

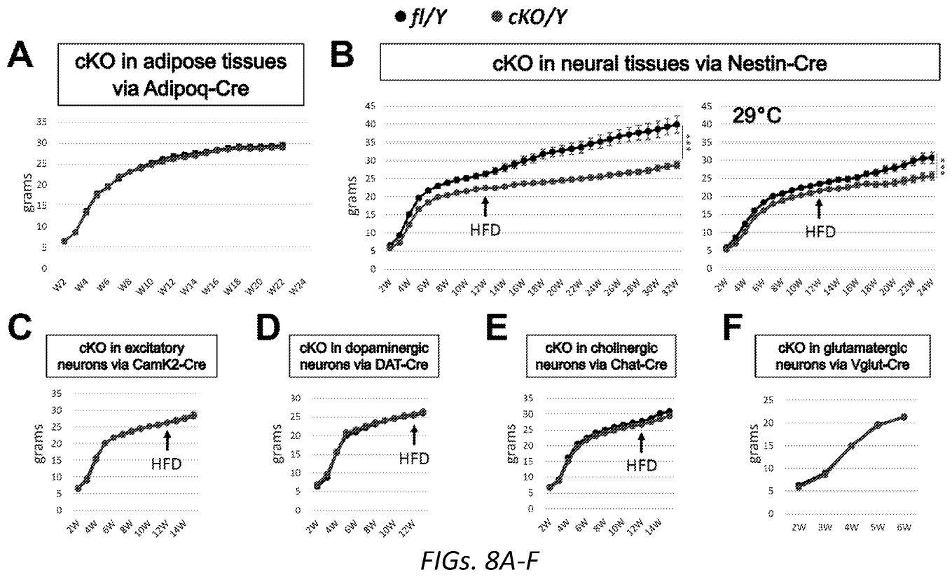

To investigate if the RlimKO phenotype was caused by an early developmental defect or by the disturbance of an ongoing, persistent activity we have targeted a systemic Rlim cKO via Ubc-Cre/ERT2, that induces tamoxifen-induced Cre expression (experiment ongoing). Indeed, inducing the cKO at 6 weeks of age, we observed the RlimKO weight phenotype was paralleled by in 6-weeks-old animals (FIG. 7B). Targeting the cKO to all adipose tissues using Adiponectin-Cre (Adipoq-Cre) (Eguchi et al., 2011) did not cause any change in animal weight (FIG. 8A). Moreover, unlike other tissues/cell types, we were unable to detect RLIM expression in adipocytes of BAT, eWAT and iWAT via IHC. Thus, Rlim regulates adipose functions acting in other cell types.

Because adipose functions are controlled by the nervous system in particular the hypothalamus (Breton, 2013; Contreras et al., 2017), we targeted the cKO to all neuronal tissues via Nestin-Cre (Tronche et al., 1999). Indeed, the Rlim cKO/Y^{Nestin-Cre} males displayed significant decreased weight when compared to fl/Y littermates (FIG. 8B), recapitulating the phenotype observed upon systemic RlimKO (FIGS. 2, 7A). Combined, these results indicate that the RlimKO phenotype was not caused by a developmental defect but by persistent activities in the nervous system, most likely in the hypothalamus.

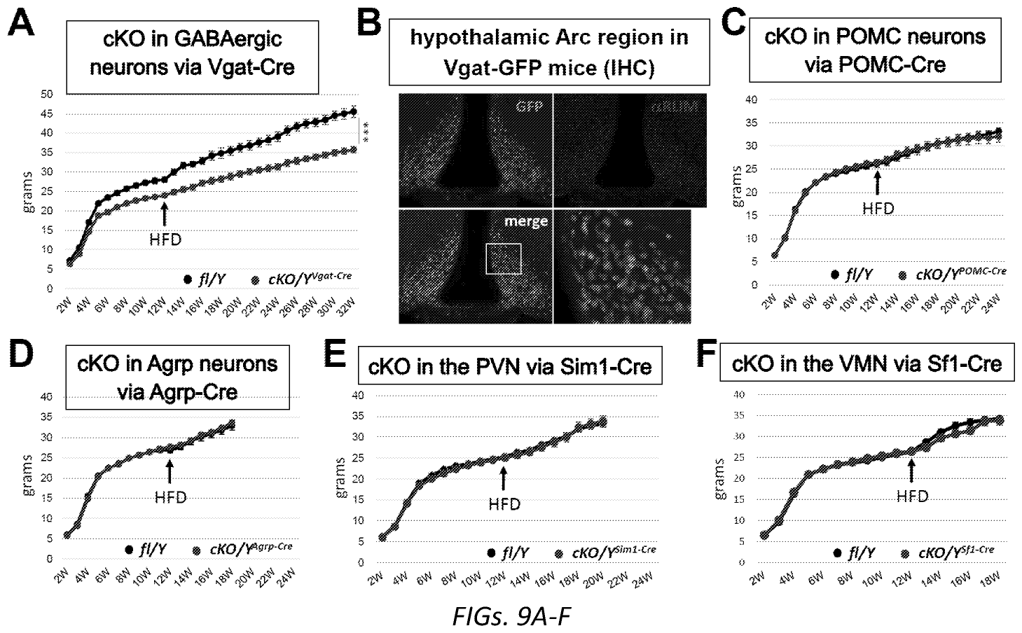

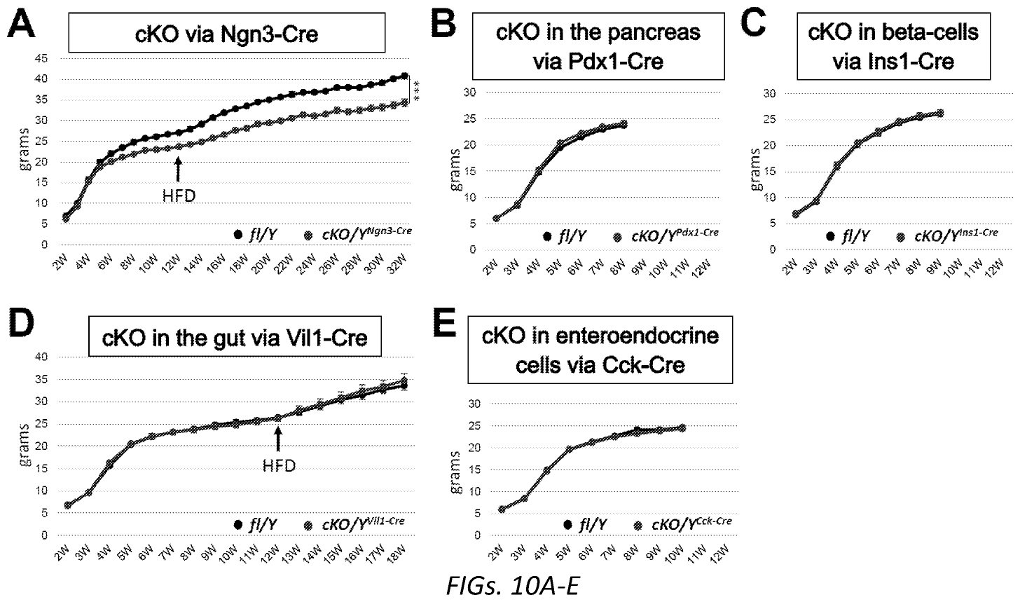

To further identify cell types/organs of Rlim action during the balancing of energy homeostasis, we have targeted the cKO via additional Cre drivers. Targeting specific neuronal subtypes, we found no major effects on animal weight by knocking out Rlim in excitatory neurons via CamK2-Cre (Tsien et al., 1996), dopaminergic neurons via DAT-Cre (Backman et al., 2006), cholinergic neurons via Chat-Cre (Rossi et al., 2011) and glutamatergic neurons via Vglut-Cre (Vong et al., 2011) (FIGS. 8C-F). In contrast, targeting the Rlim cKO to GABAergic neurons via Vgat-Cre (Vong et al., 2011) appears to recapitulate much of the weight effect (FIG. 9A). As Rlim was highly expressed in these neurons (FIG. 9B), our results suggest that Rlim in inhibitory GABAergic neurons regulates energy homeostasis. However, it appears that POMC+ or Agrp+ neurons in the Arc (FIGS. 9C, D) were not involved as well as neurons of the PVH or VMN (FIGS. 9E, F). We also found that targeting Rlim via Ngn3-Cre partially recapitulated the weight effect observed in the systemic Rlim KO (FIG. 10A). Because besides the brain the Ngn3-Cre transgene was also expressed in enteroendocrine cells in the gut as well as in pancreatic beta cells, which are also GABA+ (Franklin and Wollheim, 2004; Schonhoff et al., 2004), we excluded Rlim actions in these tissues/cell types (FIGS. 10 B-E). Thus, Rlim likely acts in distinct GABAergic neuronal subpopulations in the brain.

We have screened twelve chemically modified siRNAs of Rlim sequences conserved between mouse and human and identified seven that led to an efficient Rlim knockdown (KD) in N2A cells (FIG. 11A-B). The modification patterns, and targeting regions and sequences for all siRNAs tested are shown in Tables 1 and 2, respectively. The dioligo format of the siRNA used for brain injections is indicated in Table 3. The use of these chemically modified siRNAs will allow to modify Rlim activity in vivo thereby influencing food intake and ultimately body weight.

TABLE 1
Modification patterns of exemplary siRNAs (sense/antisense)

| Oligo ID | Sequence | No. |
|---|---|---|
| Rlim_XM_006527919_1959_s | (mA)#(mG)#(mG)(mC)(fA)(fA)(fC)(mA)(fA)(mA) | 2. |
| | (mC)(mU)(mU)#(mC)#(mA)-TegChol | |
| Rlim_XM_006527919_2724_s | (mU)#(mA)#(mU)(mU)(fU)(fA)(fU)(mC)(fU)(mU) | 3. |
| | (mG)(mU)(mG)#(mG)#(mA)-TegChol | |
| Rlim_XM_006527919_2747_s | (mA)#(mA)#(mA)(mG)(fC)(fC)(fU)(mA)(fU)(mG) | 4. |
| | (mC)(mU)(mU)#(mG)#(mA)-TegChol | |
| Rlim_XM_006527919_2772_s | (mA)#(mA)#(mA)(mA)(fU)(fC)(fA)(mC)(fA)(mU) | 5. |
| | (mU)(mC)(mA)#(mU)#(mA)-TegChol | |
| Rlim_XM_006527919_2815_s | (mA)#(mG)#(mG)(mC)(fU)(fC)(fA)(mG)(fU)(mA) | 6. |
| | (mC)(mU)(mU)#(mU)#(mA)-TegChol | |
| Rlim_XM_006527919_2831_s | (mC)#(mA)#(mA)(mU)(fG)(fC)(fA)(mC)(fU)(mG) | 7. |
| | (mU)(mU)(mG)#(mU)#(mA)-TegChol | |
| Rlim_XM_006527919_3562_s | (mC)#(mA)#(mU)(mG)(fU)(fU)(fG)(mG)(fG)(mU) | 8. |
| | (mU)(mA)(mU)#(mU)#(mA)-TegChol | |
| Rlim_XM_006527919_4070_s | (mA)#(mA)#(mG)(mG)(fA)(fA)(fA)(mU)(fA)(mA) | 9. |
| | (mU)(mG)(mU)#(mC)#(mA)-TegChol | |
| Rlim_XM_006527919_4084_s | (mC)#(mA)#(mA)(mA)(fC)(fA)(fA)(mA)(fG)(mC) | 10. |
| | (mU)(mU)(mC)#(mA)#(mA)-TegChol | |
| Rlim_XM_006527919_4137_s | (mC)#(mG)#(mC)(mU)(fU)(fA)(fU)(mG)(fU)(mA) | 11. |
| | (mA)(mU)(mA)#(mC)#(mA)-TegChol | |
| Rlim_XM_006527919_4140_s | (mU)#(mU)#(mA)(mU)(fG)(fU)(fA)(mA)(fU)(mA) | 12. |
| | (mC)(mA)(mU)#(mU)#(mA)-TegChol | |
| Rlim_XM_006527919_6849_s | (mU)#(mA)#(mG)(mG)(fU)(fA)(fC)(mG)(fC)(mA) | 13. |
| | (mG)(mU)(mU)#(mG)#(mA)-TegChol | |
| Rlim_XM_006527919_1959_as | P(mU)#(fG)#(mA)(mA)(mG)(fU)(mU)(mU)(mG)(mU)(mU) | 14. |
| | (mG)(mC)#(fC)#(mU)#(fU)#(mC)#(mU)#(mG)#(fU) | |
| Rlim_XM_006527919_2724_as | P(mU)#(fC)#(mC)(mA)(mC)(fA)(mA)(mG)(mA)(mU) | 15. |
| | (mA)(mA)(mA)#(fU)#(mA)#(fU)#(mC)#(mU)#(mC)#(fA) | |
| Rlim_XM_006527919_2747_as | P(mU)#(fC)#(mA)(mA)(mG)(fC)(mA)(mU)(mA)(mG)(mG) | 16. |
| | (mC)(mU)#(fU)#(mU)#(fU)#(mU)#(mA)#(mA)#(fU) | |
| Rlim_XM_006527919_2772_as | P(mU)#(fA)#(mU)(mG)(mA)(fA)(mU)(mG)(mU)(mG)(mA) | 17. |
| | (mU)(mU)#(fU)#(mU)#(fU)#(mU)#(mU)#(mC)#(fA) | |
| Rlim_XM_006527919_2815_as | P(mU)#(fA)#(mA)(mA)(mG)(fU)(mA)(mC)(mU)(mG)(mA) | 18. |
| | (mG)(mC)#(fC)#(mU)#(fC)#(mU)#(mA)#(mC)#(fA) | |
| Rlim_XM_006527919_2831_as | P(mU)#(fA)#(mC)(mA)(mA)(fC)(mA)(mG)(mU)(mG)(mC) | 19. |
| | (mA)(mU)#(fU)#(mG)#(fG)#(mA)#(mA)#(mA)#(fA) | |
| Rlim_XM_006527919_3562_as | P(mU)#(fA)#(mA)(mU)(mA)(fA)(mC)(mC)(mC)(mA)(mA) | 20. |
| | (mC)(mA)#(fU)#(mG)#(fA)#(mC)#(mU)#(mU)#(fC) | |
| Rlim_XM_006527919_4070_as | P(mU)#(fG)#(mA)(mC)(mA)(fU)(mU)(mA)(mU)(mU)(mU) | 21. |
| | (mC)(mC)#(fU)#(mU)#(fG)#(mC)#(mA)#(mA)#(fG) | |
| Rlim_XM_006527919_4084_as | P(mU)#(fU)#(mG)(mA)(mA)(fG)(mC)(mU)(mU)(mU)(mG) | 22. |
| | (mU)(mU)#(fU)#(mG)#(fG)#(mA)#(mC)#(mA)#(fU) | |
| Rlim_XM_006527919_4137_as | P(mU)#(fG)#(mU)(mA)(mU)(fU)(mA)(mC)(mA)(mU) | 23. |
| | (mA)(mA)(mG)#(fC)#(mG)#(fC)#(mC)#(mA)#(mU)#(fA) | |
| Rlim_XM_006527919_4140_as | P(mU)#(fA)#(mA)(mU)(mG)(fU)(mA)(mU)(mU)(mA)(mC) | 24. |
| | (mA)(mU)#(fA)#(mA)#(fG)#(mC)#(mG)#(mC)#(fC) | |
| Rlim_XM_006527919_6849_as | P(mU)#(fC)#(mA)(mA)(mC)(fU)(mG)(mC)(mG)(mU)(mA) | 25. |
| | (mC)(mC)#(fU)#(mA)#(fC)#(mU)#(mC)#(mC)#(fA) | |

No, SEQ ID NO:; Chemical modifications are designated as follows, “#”-phosphorothioate bond, “m”—2′-O-Methyl, “f”—2′-Fluoro, “P”—5′ Phosphate, “V”—5′-(E)-Vinylphosphonate. “DIO”—di-siRNA

TABLE 2
Sequence and regions of RUm Targeted by siRNA

| Oligo ID | Sequence | No. | Gene region | No. |
|---|---|---|---|---|
| Rlim_XM_ | ACAGAAGGCAACAAACUUCG | 26. | TGCATTACAGAATACACAGAA | 27. |
| 006527919_1959 | | | GGCAACAAACTTCGTAAACTACCT | |
| Rlim_XM_ | UGAGAUAUUUAUCUUGUGGA | 28. | AATTTAGAGTTTAATTGAGATA | 29. |
| 006527919_2724 | | | TTTATCTTGTGGAAATATTAAAA | |
| Rlim_XM_ | AUUAAAAAGCCUAUGCUUGU | 30. | TTATCTTGTGGAAATATTAAAA | 31. |
| 006527919_2747 | | | AGCCTATGCTTGTGTAAGTGAAA | |
| Rlim_XM_ | UGAAAAAAAUCACAUUCAUU | 32. | CTATGCTTGTGTAAGTGAAAAA | 33. |
| 006527919_2772 | | | AATCACATTCATTTGTTTAAAAA | |
| Rlim_XM_ | UGUAGAGGCUCAGUACUUUU | 34. | AATGTAAAGCTATTTTGTAGAGG | 35. |
| 006527919_2815 | | | CTCAGTACTTTTCCAATGCACT | |
| Rlim_XM_ | UUUUCCAAUGCACUGUUGUA | 36. | GTAGAGGCTCAGTACTTTTCCAA | 37. |
| 006527919_2831 | | | TGCACTGTTGTATTAATGCATT | |
| Rlim_XM_ | GAAGUCAUGUUGGGUUAUUU | 38. | ATTTTTTATTGAAAGGAAGTCATG | 39. |
| 006527919_3562 | | | TTGGGTTATTTGAGATTCAAA | |
| Rlim_XM_ | CUUGCAAGGAAAUAAUGUCC | 40. | AGGATATGTACTGCTCTTGCAAGG | 41. |
| 006527919_4070 | | | AAATAATGTCCAAACAAAGCT | |
| Rlim_XM_ | AUGUCCAAACAAAGCUUCAC | 42. | TCTTGCAAGGAAATAATGTCCAAA | 43. |
| 006527919_4084 | | | CAAAGCTTCACTTATTTTTTT | |
| Rlim_XM_ | UAUGGCGCUUAUGUAAUACA | 44. | GCATATTTCACTGTTTATGGCGCTT | 45. |
| 006527919_4137 | | | ATGTAATACATTTTTATCTT | |
| Rlim_XM_ | GGCGCUUAUGUAAUACAUUU | 46. | TATTTCACTGTTTATGGCGCTTATGT | 47. |
| 006527919_4140 | | | AATACATTTTTATCTTTTT | |
| Rlim_XM_ | UGGAGUAGGUACGCAGUUGG | 48. | CTGTGAAGTGTCCAGTGGAGTAGGT | 49. |
| 006527919_6849 | | | ACGCAGTTGGCCTGGTTATA | |

### No., SEQ ID NO:

TABLE 3
Exemplary sequences in divalent chemistry (P3 m/f pattern):

| Oligo | Sequence | No. |
|---|---|---|
| RLIM_4070_p3_AS | VP(mU)#(fG)#(mA)(fC)(fA)(fU)(mU)(fA)(mU)(fU)(mU)(fC)(mC) | 50. |
| | (fU)#(mU)#(fG)#(mC)#(mA)#(mA)#(fG)#(mU) | |
| RLIM_4070_p3_S_Dio | (mC)#(mA)#(mA)(fG)(mG)(fA)(mA)(fA)(mU)(fA)(mA)(mU)(mG) | 51. |
| | (fU)#(mC)#(mA)-Dio | |

No., SEQ ID NO:; Detailed sequence and chemical modification patterns of siRNAs. Chemical modifications are designated as follows, “#”-phosphorothioate bond, “m”—2′-O-Methyl, “f”-2′-Fluoro, “P”—5′ Phosphate, “V”—5′-(E)-Vinylphosphonate. “DIO”—di-siRNA

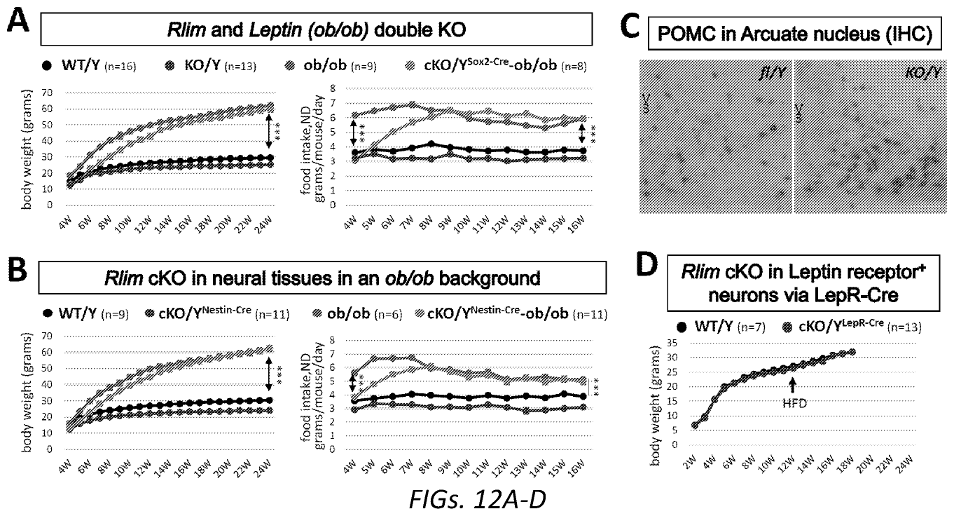

Because GABAergic neurons mediate Leptin action to prevent obesity (Vong et al., 2011), we generated male mice systemically lacking Rlim in an ob/ob background. ob/ob mice develop obesity due to excessive appetite caused by mutation of the leptin gene (Zhang et al., 1994). Comparisons of body weight show that ob/ob males display significantly higher body weight at weaning when compared to WT, KO and doubleKO (dKO) males (FIG. 12A, left panel). However, in contrast to WT and KO males, the weight in dKO animals catches up to that of dKO males over time until there was no statistical difference from 15 weeks onwards. Consistent with this are the data on food intake (FIG. 12A, right panel): While ob/ob males eat considerably more than WT, KO and dKO males at 4 weeks of age, the food intake of dKO adjusts after weaning to that of ob/ob mice at 9 weeks of age. Thus, during lactation the effect of ob/ob on food intake was fully dependent on Rlim. In adults, however, no effect of the Rlim KO in the ob/ob background was observed both on body weight and food intake, indicating that Rlim acts downstream in the leptin pathway. These data were confirmed in animals with an Rlim cKO^{Nestin-Cre} in an ob/ob background (FIG. 12B). Consistent with the fact that leptin action reduces the inhibitory tone to POMC neurons via GABAergic neurons (Vong et al., 2011), IHC staining of adult mouse brains (32 weeks; HFD) revealed increased levels of POMC in hypothalamic Arcuate nuclei of animals lacking Rlim (FIG. 12C). Moreover, targeting the Rlim cKO to leptin receptor⁺ neurons via LepR-Cre (DeFalco et al., 2001) reveals no effects on body weight (FIG. 12D), indicating functions of Rlim in cell types different from leptin receptor-positive neurons. These results are overall consistent with the published literature and 1) uncover a major shift in the regulation of food intake by the leptin pathway after weaning, 2) establish a functional link between Rlim and leptin genes in the regulation of food intake and 3) indicate that functions of Rlim in the leptin pathway are likely in second or higher order neurons.

We use siRNA-mediated silencing of Rlim in mice not only to examine whether the interference of Rlim in the brain causes the observed phenotype, but also to provide evidence that interference with the Rlim pathway protects from obesity. This is carried out via stereotactic injections of chemically modified siRNAs with a divalent chemical scaffold into the 3^{rd} ventricle in the brain (Alterman et al., 2019), aimed to silence endogenous Rlim in the CNS of mice wild-type for Rlim.

### REFERENCES

Alterman, J. F., et al. (2019). A divalent siRNA chemical scaffold for potent and sustained modulation of gene expression throughout the central nervous system. Nat Biotechnol. 37, 884-894.
Bach, I., Rodriguez-Esteban, C., Carriere, C., Bhushan, A., Krones, A., Rose, D. W., Glass, C. K., Andersen, B., Izpisua Belonte, J. C., and Rosenfeld, M. G. (1999). RLIM inhibits functional activity of LIM homeodomain transcription factors via recruitment of the histone deacetylase complex. Nat Genet 22, 394-399.
Backman, C. M., Malik, N., Zhang, Y., Shan, L., Grinberg, A., Hoffer, B. J., Westphal, H., and Tomac, A. C. (2006). Characterization of a mouse strain expressing Cre recombinase from the 3′ untranslated region of the dopamine transporter locus. Genesis 44, 383-390.
Bartness, T. J., Liu, Y., Shrestha, Y. B., and Ryu, V. (2014). Neural innervation of white adipose tissue and the control of lipolysis. Front Neuroendocrinol 35, 473-493.
Bartness, T. J., Vaughan, C. H., and Song, C. K. (2010). Sympathetic and sensory innervation of brown adipose tissue. Int J Obes (Lond) 34 Suppl 1, S36-42.
Becker, T., Bossenz, M., Tursun, B., Schluter, A., Peters, M. A., Becker, C. G., Ostendorff, H. P., and Bach, I. (2003). Comparing protein stabilities during zebrafish embryogenesis. Methods in cell science. an official journal of the Society for In Vitro Biology 25, 85-89.
Biscans, A., Coles, A., Haraszti, R., Echeverria, D., Hassler, M., Osborn, M., and Khvorova, A. (2018). Diverse lipid conjugates for functional extra-hepatic siRNA delivery in vivo. Nucleic acids research 47, 1082-1096.
Breton, C. (2013). The hypothalamus-adipose axis is a key target of developmental programming by maternal nutritional manipulation. The Journal of endocrinology 216, R19-31.
Contreras, C., Nogueiras, R., Dieguez, C., Rahmouni, K., and Lopez, M. (2017). Traveling from the hypothalamus to the adipose tissue: The thermogenic pathway. Redox Biol 12, 854-863.
Cristancho, A. G., and Lazar, M. A. (2011). Forming functional fat: a growing understanding of adipocyte differentiation. Nature reviews Molecular cell biology 12, 722-734.
Cypess, A. M., Weiner, L. S., Roberts-Toler, C., Franquet Elia, E., Kessler, S. H., Kahn, P. A., English, J., Chatman, K., Trauger, S. A., Doria, A., et al. (2015). Activation of human brown adipose tissue by a beta3-adrenergic receptor agonist. Cell Metab 21, 33-38.
DeFalco, J., Tomishima, M., Liu, H., Zhao, C., Cai, X., Marth, J. D., Enquist, L., and Friedman, J. M. (2001). Virus-assisted mapping of neural inputs to a feeding center in the hypothalamus. Science 291, 2608-2613.
Dietrich, M. O., and Horvath, T. L. (2012). Limitations in anti-obesity drug development: the critical role of hunger-promoting neurons. Nat Rev Drug Discov 11, 675-691.
Disteche, C. M. (2012). Dosage compensation of the sex chromosomes. Annual review of genetics 46, 537-560.
Eguchi, J., Wang, X., Yu, S., Kershaw, E. E., Chiu, P. C., Dushay, J., Estall, J. L., Klein, U., Maratos-Flier, E., and Rosen, E. D. (2011). Transcriptional control of adipose lipid handling by IRF4. Cell Metab 13, 249-259.
Fischer, A. W., Cannon, B., and Nedergaard, J. (2018). Optimal housing temperatures for mice to mimic the thermal environment of humans: An experimental study. Mol Metab 7, 161-170.
Franklin, I. K. & Wollheim, C. B. GABA in the endocrine pancreas: its putative role as an islet cell paracrine-signalling molecule. (2004) J Gen Physiol 123, 185-190.
Gontan, C., Achame, E. M., Demmers, J., Barakat, T. S., Rentmeester, E., van, I. W., Grootegoed, J. A., and Gribnau, J. (2012). RNF12 initiates X-chromosome inactivation by targeting REX1 for degradation. Nature 485, 386-390.
Gungor, C., Taniguchi-Ishigaki, N., Ma, H., Drung, A., Tursun, B., Ostendorff, H. P., Bossenz, M., Becker, C. G., Becker, T., and Bach, I. (2007). Proteasomal selection of multiprotein complexes recruited by LIM homeodomain transcription factors. Proceedings of the National Academy of Sciences of the United States of America 104, 15000-15005.
Hayashi, S., Lewis, P., Pevny, L., and McMahon, A. P. (2002). Efficient gene modulation in mouse epiblast using a Sox2Cre transgenic mouse strain. Mechanisms of development 119 Suppl 1, S97-S101.
Hayashi, S., Tenzen, T., and McMahon, A. P. (2003). Maternal inheritance of Cre activity in a Sox2Cre deleter strain. Genesis 37, 51-53.
Ibrahim, M. M. (2010). Subcutaneous and visceral adipose tissue: structural and functional differences. Obes Rev 11, 11-18.
Jespersen, N. Z., Larsen, T. J., Peijs, L., Daugaard, S., Homoe, P., Loft, A., de Jong, J., Mathur, N., Cannon, B., Nedergaard, J., et al. (2013). A classical brown adipose tissue mRNA signature partly overlaps with brite in the supraclavicular region of adult humans. Cell Metab 17, 798-805.
Jiao, B., Ma, H., Shokhirev, M. N., Drung, A., Yang, Q., Shin, J., Lu, S., Byron, M., Kalantry, S., Mercurio, A. M., et al. (2012). Paternal RLIM/Rnf12 is a survival factor for milk-producing alveolar cells. Cell 149, 630-641.
Jiao, B., Taniguchi-Ishigaki, N., Gungor, C., Peters, M. A., Chen, Y. W., Riethdorf, S., Drung, A., Ahronian, L. G., Shin, J., Pagnis, R., et al. (2013). Functional activity of RLIM/Rnf12 is regulated by phosphorylation-dependent nucleocytoplasmic shuttling. Molecular biology of the cell 24, 3085-3096.
Johnsen, S. A., Gungor, C., Prenzel, T., Riethdorf, S., Riethdorf, L., Taniguchi-Ishigaki, N., Rau, T., Tursun, B., Furlow, J. D., Sauter, G., et al. (2009). Regulation of estrogen-dependent transcription by the LIM cofactors CLIM and RLIM in breast cancer. Cancer research 69, 128-136.
Kramer, O. H., Zhu, P., Ostendorff, H. P., Golebiewski, M., Tiefenbach, J., Peters, M. A., Brill, B., Groner, B., Bach, I., Heinzel, T., et al. (2003). The histone deacetylase inhibitor valproic acid selectively induces proteasomal degradation of HDAC2. The EMBO journal 22, 3411-3420.
Lee, J. T., and Bartolomei, M. S. (2013). X-inactivation, imprinting, and long noncoding RNAs in health and disease. Cell 152, 1308-1323.
Morton, G. J., Cummings, D. E., Baskin, D. G., Barsh, G. S., and Schwartz, M. W. (2006). Central nervous system control of food intake and body weight. Nature 443, 289-295.
Morton, G. J., Meek, T. H., and Schwartz, M. W. (2014). Neurobiology of food intake in health and disease. Nat Rev Neurosci 15, 367-378.
Ostendorff, H. P., Bossenz, M., Mincheva, A., Copeland, N. G., Gilbert, D. J., Jenkins, N. A., Lichter, P., and Bach, I. (2000). Functional characterization of the gene encoding RLIM, the corepressor of LIM homeodomain factors. Genomics 69, 120-130.
Ostendorff, H. P., Peirano, R. I., Peters, M. A., Schluter, A., Bossenz, M., Scheffner, M., and Bach, I. (2002). Ubiquitination-dependent cofactor exchange on LIM homeodomain transcription factors. Nature 416, 99-103.
Ostendorff, H. P., Tursun, B., Cornils, K., Schluter, A., Drung, A., Gungor, C., and Bach, I. (2006). Dynamic expression of LIM cofactors in the developing mouse neural tube. Developmental dynamics: an official publication of the American Association of Anatomists 235, 786-791.
Payer, B. (2016). Developmental regulation of X-chromosome inactivation. Seminars in cell & developmental biology 56, 88-99.
Rossi, J., Balthasar, N., Olson, D., Scott, M., Berglund, E., Lee, C. E., Choi, M. J., Lauzon, D., Lowell, B. B., and Elmquist, J. K. (2011). Melanocortin-4 receptors expressed by cholinergic neurons regulate energy balance and glucose homeostasis. Cell Metab 13, 195-204.
Ruzankina, Y., Pinzon-Guzman, C., Asare, A., Ong, T., Pontano, L., Cotsarelis, G., Zediak, V. P., Velez, M., Bhandoola, A., and Brown, E. J. (2007). Deletion of the developmentally essential gene ATR in adult mice leads to age-related phenotypes and stem cell loss. Cell stem cell 1, 113-126.
Schonhoff, S. E., Giel-Moloney, M. & Leiter, A. B. (2004). Minireview: Development and differentiation of gut endocrine cells. Endocrinology 145, 2639-2644.
Shin, J., Bossenz, M., Chung, Y., Ma, H., Byron, M., Taniguchi-Ishigaki, N., Zhu, X., Jiao, B., Hall, L. L., Green, M. R., et al. (2010). Maternal Rnf12/RLIM is required for imprinted X-chromosome inactivation in mice. Nature 467, 977-981.
Shin, J., Wallingford, M. C., Gallant, J., Marcho, C., Jiao, B., Byron, M., Bossenz, M., Lawrence, J. B., Jones, S. N., Mager, J., et al. (2014). RLIM is dispensable for X-chromosome inactivation in the mouse embryonic epiblast. Nature 511, 86-89.
Tronche, F., Kellendonk, C., Kretz, O., Gass, P., Anlag, K., Orban, P. C., Bock, R., Klein, R., and Schutz, G. (1999). Disruption of the glucocorticoid receptor gene in the nervous system results in reduced anxiety. Nature genetics 23, 99-103.
Tsien, J. Z., Chen, D. F., Gerber, D., Tom, C., Mercer, E. H., Anderson, D. J., Mayford, M., Kandel, E. R., and Tonegawa, S. (1996). Subregion- and cell type-restricted gene knockout in mouse brain. Cell 87, 1317-1326.
Vong, L., Ye, C., Yang, Z., Choi, B., Chua, S., Jr., and Lowell, B. B. (2011). Leptin action on GABAergic neurons prevents obesity and reduces inhibitory tone to POMC neurons. Neuron 71, 142-154.
Wang, F., Gervasi, M. G., Boskovid, A., Sun, F., Rinaldi, V. D., Yu, J., Wallingford, M. C., Tourzani, D. A., Mager, J., Zhu, L. J., et al. (2020). Deficient spermiogenesis in mice lacking Rlim. https://doi.org/10.1101/2020.08.31.275248
Wang, F., Shin, J., Shea, J. M., Yu, J., Boskovic, A., Byron, M., Zhu, X., Shalek, A. K., Regev, A., Lawrence, J. B., et al. (2016). Regulation of X-linked gene expression during early mouse development by Rlim. Elife 5.
Wu, J., Bostrom, P., Sparks, L. M., Ye, L., Choi, J. H., Giang, A. H., Khandekar, M., Virtanen, K. A., Nuutila, P., Schaart, G., et al. (2012). Beige adipocytes are a distinct type of thermogenic fat cell in mouse and human. Cell 150, 366-376.
Zhang, Y., Proenca, R., Maffei, M., Barone, M., Leopold, L., and Friedman, J. M. (1994). Positional cloning of the mouse obese gene and its human homologue. Nature 372, 425-432.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating, or reducing risk of, obesity or a disorder associated with obesity, or improving glycemic control, in a mammalian subject, the method comprising administering a therapeutically effective amount of an inhibitory nucleic acid targeting Rlim to a subject in need thereof.

2. The method of claim 1, wherein the disorder associated with obesity is diabetes, metabolic syndrome, fatty liver disease, non-hepatic steatosis.

3. The method of claim 1, wherein the inhibitory nucleic acid is an antisense, siRNA, or shRNA.

4. The method claim 1, wherein the inhibitory nucleic acid is an siRNA comprising at least 10 consecutive nucleotides of SEQ ID NO: 2-25, 26, 28, 30, 32, 34, 36, 38, 40, 42, 46, 48, 50, or 51.

5. The method of claim 1, wherein the inhibitory nucleic acid comprises one or more modified bonds or bases.

6. The method of claim 5, wherein the one or more modified bonds or bases comprise morpholinos, phosphorothioate backbones, peptide nucleic acid (PNA), or locked nucleic acid (LNA) molecules.

7. The method of claim 1, wherein the inhibitory nucleic acid is conjugated to a N-Acetylgalactosamine (GalNAc) and/or hydrophobic moiety.

8. The method of claim 7, wherein the hydrophobic moiety is or comprises dichloroacetic acid (DCA) or Phosphatidylcholine (PC)-DCA, Docosahexaenoic acid (DHA), or Phosphatidylcholine-DHA (g2DHA), or cholesterol.

9. The method of claim 1, wherein the inhibitory nucleic acid is divalent, trivalent, or tetravalent.

10. The method of claim 1, wherein the subject has a BMI of at least 25.

11. The method of claim 1, wherein the subject is human.

12. A method of treating, or reducing risk of, underweight or a disorder associated with underweight in a mammalian subject, the method comprising administering a therapeutically effective amount of an Rlim polypeptide to a subject in need thereof.

13. The method of claim 12, wherein the subject has a BMI of less than 18.5.

14. The method of claim 12, wherein the subject is human.

15. The method of claim 14, comprising administering (i) a polypeptide comprising a sequence that is at least 95% identical to SEQ ID NO:1, or an active fragment thereof, or (ii) a nucleic acid encoding a polypeptide comprising a sequence that is at least 95% identical to SEQ ID NO:1, or an active fragment thereof.

16. The method of claim 15, comprising administering a nucleic acid encoding a polypeptide comprising a sequence that is at least 95% identical to SEQ ID NO:1, in a viral vector.

17. The method of claim 16, wherein the viral vector is an adeno-associated viral (AAV) vector.

18. The method of claim 1, wherein the inhibitory nucleic acid is administered parenterally, optionally intravenously, intramuscularly, or subcutaneously.

19. (canceled)

20. The method of claim 12, wherein the polypeptide comprises one or more modifications.

21. The method of claim 20, wherein the modification comprises one or more of: replacement of one or more L amino acids with D amino acids; acetylation, amidation; conjugation to a linear or branched-chain monomethoxy poly-ethylene glycol (PEG); modification of the N- or C-terminus; glycosylation; polysialic acid (PSA) addition to a glycan; or fusion to a non-Rlim protein.

22. An inhibitory nucleic acid comprising at least 10 consecutive nucleotides of SEQ ID NO: 2-25, 26, 28, 30, 32, 34, 36, 38, 40, 42, 46, 48, 50, or 51, wherein the inhibitory nucleic acid comprises one or more modified bonds or bases.

23. (canceled)

24. The inhibitory nucleic acid of claim 22, wherein the one or more modified bonds or bases comprise morpholinos, phosphorothioate backbones, peptide nucleic acid (PNA), or locked nucleic acid (LNA) molecules.

25. The inhibitory nucleic acid of claim 22, wherein the inhibitory nucleic acid is conjugated to a N-Acetylgalactosamine (GalNAc) and/or a hydrophobic moiety.

26. The inhibitory nucleic acid of claim 25, wherein the hydrophobic moiety is or comprises dichloroacetic acid (DCA) or Phosphatidylcholine (PC)-DCA, Docosahexaenoic acid (DHA), or Phosphatidylcholine-DHA (g2DHA), or cholesterol.

27. The inhibitory nucleic acid of claim 25, wherein the inhibitory nucleic acid is divalent, trivalent, or tetravalent.

28.-31. (canceled)
